SEQUENCE LISTING (39 sequences)

SEQ ID NO: 1 - protein, 494 aa, Salmonella typhimurium
AQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 2 - protein, 505 aa, Salmonella muenchen
AQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYALKMGDDFYAATYDEKTGTITAKTTTYTDGAGVAQTGAVKFGGANGKSEVVTATDGKTYLASDLDKHNFRTGGELKEVNTDKTENPLQKIDAALAQVDALRSDLGAVQNRFNSAITNLGNTVNNLSSARSRIEDSDYATEVSNMSRAQILQQAGTSVLAQANQVPQNVLSLLR

SEQ ID NO: 3 - protein, 553 aa, Escherichia coli
AQVINTNSLSLITQNNINKNQSALSSSIERLSSGLRINSAKDDAAGQAIANRFTSNIKGLTQAARNANDGISVAQTTEGALSEINNNLQRIRELTVQATTGTNSDSDLDSIQDEIKSRLDEIDRVSGQTQFNGVNVLSKDGSMKIQVGANDGETITIDLKKIDSDTLNLAGFNVNGEGETANTAATLKDMVGLKLDNTGVTTAGVNRYIADKAVASSTDILNAVAGVDGSKVSTEADVGFGAAAPGTPVEYTYHKDTNTYTASASVDATQLAAFLNPEAGGTTAATVSIGNGTTAQEQKVIIAKDGSLTAADDGAALYLDDTGNLSKTNAGTDTQAKLSDLMANNANAKTVITTDKGTFTANTTKFDGVDISVDASTFANAVKNETYTATVGVTLPATYTVNNGTAASAYLVDGKVSKTPAEYFAQADGTITSGENAATSKAIYVSANGNLTTNTTSESEATTNPLAALDDAIASIDKFRSSLGAIQNRLDSAVTNLNNTTTNLSEAQSRIQDADYATEVSNMSKAQIIQQAGNSVLAKANQVPQQVLSLQQG

SEQ ID NO: 4 - protein, 14 aa, Salmonella muenchen
AVQSANGTNSQSDL

SEQ ID NO: 5 - protein, 172 aa, Salmonella muenchen
AQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKL

SEQ ID NO: 6 - protein, 9 aa, Salmonella muenchen
NRFNSAITN

SEQ ID NO: 7 - protein, 88 aa, Salmonella muenchen
PLQKIDAALAQVDALRSDLGAVQNRFNSAITNLGNTVNNLSSARSRIEDSDYATEVSNMSRAQILQQAGTSVLAQANQVPQNVLSLLR

SEQ ID NO: 8 - protein, 376 aa, Vibrio vulnificus
MAINVNTNVSAMTAQRYLNQAAEGQQKSMERLSSGYKINSAKDDAAGLQISNRLNSQSRGLDMAVKNANDGISIAQTAEGAMTETTNILQRMRDLALQSSNGSNSRSERVAIQEEVSALNQELNRIAETTSFGGNKLLNGTYGSQSFQIGADSGEAVMLSMGNLRSDTDAMGGLSYKSEEGVGADWRVSGNTDFTMSYVNKQGEEKEITVNAKAGDDLEELATYINGQNDDVKASVGEGGKLQLFASNQRVEGEVEFGGGLASELNIGDGTKTNVSNIDVTTVAGSQEAVSIIDGALKSVDSERASLGAFQNRFNHAISNLSNINENVNASSSRIKDTDYAKETTQMTKTQILQQASTSILAQAKQSPSAALSLLG

SEQ ID NO: 9 - protein, 162 aa, Salmonella typhimurium
GISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDKIDAALAQVDTLRSDLGAVQNRFNSAITNLGNTVNNLTSARSRIEDSDYATEVSNMSRAQI

SEQ ID NO: 10 - protein, 165 aa, Salmonella typhimurium
GISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNKIDAALAQVDTLRSDLGAVQNRFNSAITNLGNTVNNLTSARSRIEDSDYATEVSNMSRAQI

SEQ ID NO: 11 - protein, 171 aa, Salmonella typhimurium
GISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDATTTENPLQKIDAALAQVDTLRSDLGAVQNRFNSAITNLGNTVNNLTSARSRIEDSDYATEVSNMSRAQI

SEQ ID NO: 12 - protein, 186 aa, Salmonella typhimurium
GISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 13 - protein, 174 aa, Salmonella typhimurium
GISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNATTTENPLQKIDAALAQVDTLRSDLGAVQNRFNSAITNLGNTVNNLTSARSRIEDSDYATEVSNMSRAQI

SEQ ID NO: 14 - protein, 189 aa, Salmonella typhimurium
GISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 15 - protein, 195 aa, Salmonella typhimurium
GISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 16 - protein, 198 aa, Salmonella typhimurium
GISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 17 - protein, 167 aa, Salmonella typhimurium
RINSAGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDKIDAALAQVDTLRSDLGAVQNRFNSAITNLGNTVNNLTSARSRIEDSDYATEVSNMSRAQI

SEQ ID NO: 18 - protein, 170 aa, Salmonella typhimurium
RINSAGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNKIDAALAQVDTLRSDLGAVQNRFNSAITNLGNTVNNLTSARSRIEDSDYATEVSNMSRAQI

SEQ ID NO: 19 - protein, 176 aa, Salmonella typhimurium
RINSAGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDATTTENPLQKIDAALAQVDTLRSDLGAVQNRFNSAITNLGNTVNNLTSARSRIEDSDYATEVSNMSRAQI

SEQ ID NO: 20 - protein, 191 aa, Salmonella typhimurium
RINSAGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 21 - protein, 179 aa, Salmonella typhimurium
RINSAGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNATTTENPLQKIDAALAQVDTLRSDLGAVQNRFNSAITNLGNTVNNLTSARSRIEDSDYATEVSNMSRAQI

SEQ ID NO: 22 - protein, 194 aa, Salmonella typhimurium
RINSAGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 23 - protein, 200 aa, Salmonella typhimurium
RINSAGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 24 - protein, 203 aa, Salmonella typhimurium
RINSAGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 25 - protein, 182 aa, Salmonella typhimurium
ANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDKIDAALAQVDTLRSDLGAVQNRFNSAITNLGNTVNNLTSARSRIEDSDYATEVSNMSRAQI

SEQ ID NO: 26 - protein, 185 aa, Salmonella typhimurium
ANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNKIDAALAQVDTLRSDLGAVQNRFNSAITNLGNTVNNLTSARSRIEDSDYATEVSNMSRAQI

SEQ ID NO: 27 - protein, 191 aa, Salmonella typhimurium
ANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDATTTENPLQKIDAALAQVDTLRSDLGAVQNRFNSAITNLGNTVNNLTSARSRIEDSDYATEVSNMSRAQI

SEQ ID NO: 28 - protein, 206 aa, Salmonella typhimurium
ANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 29 - protein, 194 aa, Salmonella typhimurium
ANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNATTTENPLQKIDAALAQVDTLRSDLGAVQNRFNSAITNLGNTVNNLTSARSRIEDSDYATEVSNMSRAQI

SEQ ID NO: 30 - protein, 209 aa, Salmonella typhimurium
ANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 31 - protein, 215 aa, Salmonella typhimurium
ANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 32 - protein, 218 aa, Salmonella typhimurium
ANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 33 - protein, 490 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTBD

SEQ ID NO: 34 - protein, 734 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDWTWILFLVAAATRVHSRINSAGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNGAAGATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 35 - protein, 1022 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDWTWILFLVAAATRVHSMAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 36 - protein, 4 aa, Artificial Sequence
  Synthetic Construct
GAAG

SEQ ID NO: 37 - protein, 2 aa, Artificial Sequence
  Synthetic Construct
  VARIANT (1)..(2): Can be present in repeats of any integer up to 10
GS

SEQ ID NO: 38 - protein, 5 aa, Artificial Sequence
  Synthetic Construct
  VARIANT (1)..(5): Can be present in repeats of any integer up to 10
GSGGS

SEQ ID NO: 39 - protein, 4 aa, Artificial Sequence
  Synthetic Construct
  VARIANT (1)..(4): Can be present in repeats of any integer up to 10
GGGS

(19) United States
(12) Patent Application Publication
(10) Pub. No.: US 2022/0096547 A1
(43) Pub. Date: Mar. 31, 2022

(54) MODIFIED IMMUNE CELLS EXPRESSING FLAGELLIN POLYPEPTIDE

(71) Applicant: Nanjing Legend Biotech Co., Ltd., Nanjing, Jiangsu (CN)

(72) Inventors: Yafeng ZHANG, Nanjing, Jiangsu (CN); Shu WU, Nanjing, Jiangsu (CN); Zhongyuan TU, Nanjing, Jiangsu (CN); Wang ZHANG, Nanjing, Jiangsu (CN); Qinghe ZHANG, Nanjing, Jiangsu (CN)

(21) Appl. No.: 17/420,657

(22) PCT Filed: Jan. 3, 2020
(86) PCT No.: PCT/CN2020/070282

§ 371 Date: Jul. 2, 2021

(30) Foreign Application Priority Data
Jan. 3, 2019 (CN) PCT/CN2019/070296

(51) Int. Cl. A61K 35/17; C07K 14/255; C12N 15/63; C12N 5/0783; C07K 14/725; C07K 14/705; C12N 5/16

(52) U.S. Cl. CPC A61K 35/17; C07K 14/255; C12N 15/63; C12N 5/0636; A61K 2039/505; C07K 14/70521; C07K 14/70578; C07K 14/70596; C12N 5/163; C07K 14/7051

(57) ABSTRACT

Provided are modified immune cells expressing a flagellin polypeptide capable of binding to a toll-like receptor. The modified immune cell further comprises an engineered receptor. Also provided are methods and pharmaceutical compositions for cancer treatment using the modified immune cells.

### CROSS REFERENCE TO RELATED APPLICATIONS

This application claims priority benefit of International Patent Application No. PCT/CN2019/070296 filed on Jan. 3, 2019, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761422001841.txt, date recorded: Dec. 28, 2019, size: 76 KB).

### FIELD OF THE INVENTION

The present invention relates to modified immune cells that express a flagellin polypeptide, and methods of use thereof for treating cancer.

### BACKGROUND OF THE INVENTION

Cancer vaccines aim to elicit immune response against tumor antigens that are exclusively expressed in tumor cells (e.g., cancer testes antigens, mutated proteins, and viral antigens), or expressed at an elevated level (e.g., overexpressed or differentially expressed) in tumor cells. A variety of cancer vaccines have been tested clinically, including peptide vaccines, plasmid DNA vaccines, RNA vaccines, dendritic cells (DCs) and T cells. T cells modified to express tumor antigen peptides can elicit strong, durable responses in animals and humans, and adoptively transferred T cells can migrate efficiently to secondary lymphoid organs when antigen priming occurs. However, cancer cells have mechanisms to escape immune surveillance, which compromise the efficacy of T cell based vaccines. For example, the microenvironment of cancer cells may result in inefficient T cell priming, immune tolerance, or immunosuppression by regulatory T cells (Tregs). There remains a need for highly efficient cell-based cancer immunotherapy.

Flagellin is a subunit protein of the flagellum, a whip-like appendage that enables bacterial motility. Recent studies have shown that flagellin is a potent activator of pro-inflammatory eukaryotic cell signaling via its interaction with Toll-like Receptor (TLR) 5. Flagellin regulates both the innate and adaptive arms of immunity during microbial infections. Flagellin stimulates the production of pro-inflammatory cytokines and chemokines in a number of innate and non-immune cells, including dendritic cells (DCs), Natural Killer (NK) cells, epithelial cells, and lymph node stromal cells. Flagellin can stimulate T cell proliferation both directly and by recruiting innate immune cells to the site of infection. However, under certain circumstances, flagellin may also enhance the immuno-suppressive capacity of CD4⁺CD25⁺ Tregs. Flagellin has been used as adjuvants in vaccines, including anti-cancer vaccines in combination with tumor antigen peptides. Furthermore, recombinant flagellin and Salmonella typhimurium secreting Vibrio vulnificus flagellin have shown anti-tumor activities in animal models. However, administration of purified flagellin at the time of tumor transplantation enhanced tumor growth. The expression of TLR5 has been show to increase in various types of cancers, such as in gastric and colorectal cancers. See, Hajam I. A. et al. Experimental & Molecular Medicine (2017) 49: e373.

The disclosures of all publications, patents, patent applications and published patent applications referred to herein are hereby incorporated herein by reference in their entirety.

### BRIEF SUMMARY OF THE INVENTION

The present application provides modified immune cells that express a flagellin polypeptide that is capable of binding to a toll-like receptor (e.g., TLR5), and methods of use thereof for treating cancer.

One aspect of the present application provides a modified immune cell comprising a first heterologous nucleic acid sequence encoding a flagellin polypeptide comprising a flagellin protein or a fragment thereof, wherein the flagellin polypeptide upon expression is capable of binding to a toll-like receptor. In some embodiments, the flagellin polypeptide comprises Motif N of a flagellin protein. In some embodiments, the flagellin polypeptide comprises Motif C of a flagellin protein. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises all or a portion of an amino acid sequence having at least about 85% sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2 or SEQ ID NO: 3.

In some embodiments according to any one of the modified immune cells described above, the flagellin polypeptide comprises an amino acid sequence having at least about 85% sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the flagellin polypeptide comprises an amino acid sequence having at least about 85% sequence identity to SEQ ID NO: 24. In some embodiments, the flagellin polypeptide comprises the amino acid sequence of SEQ ID NO: 24. In some embodiments, the flagellin polypeptide comprises an amino acid sequence having at least about 85% sequence identity to SEQ ID NO: 32. In some embodiments, the flagellin polypeptide comprises the amino acid sequence of SEQ ID NO: 32.

In some embodiments according to any one of the modified immune cells described above, the toll-like receptor is selected from the group consisting of TLR4, TLR5, TLR11, TLR2, TLR5, and TLR5. In some embodiments, the flagellin polypeptide is capable of binding to TLR5, such as a TLR5 homodimer or a TLR4/TLR5 heterodimer. In some embodiments, the flagellin polypeptide is capable of binding to TLR11.

In some embodiments according to any one of the modified immune cells described above, the flagellin polypeptide is membrane-bound. In some embodiments, the flagellin polypeptide is bound to the cell membrane via a glycosylphosphatidylinositol (GPI) linker. In some embodiments, the flagellin polypeptide is bound to the cell membrane via a transmembrane domain. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge region, such as a CD8 hinge region. In some embodiments, the flagellin polypeptide further comprises an intracellular signaling domain. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, Ligands of CD83 and combinations thereof. In some embodiments, the flagellin polypeptide is secreted by the modified immune cell.

In some embodiments according to any one of the modified immune cells described above, the modified immune cell is selected from the group consisting of a cytotoxic T cell, a helper T cell, a natural killer (NK) cell, an NK-T cell, an iNK-T cell, an NK-T like cell, an αβT cell and a γδT cell. In some embodiments, the modified immune cell is a cytotoxic T cell. In some embodiments, the modified immune cell is a γδT cell. In some embodiments, the modified immune cell is a tumor-infiltrating T cell or DC-activated T cell.

In some embodiments according to any one of the modified immune cells described above, the modified immune cell comprises a second heterologous nucleic acid sequence encoding an engineered receptor. In some embodiments, the engineered receptor is a chimeric antigen receptor (CAR). In some embodiments, the CAR is an anti-BCMA CAR. In some embodiments, the engineered receptor is a modified T-cell receptor (TCR). In some embodiments, the engineered receptor is a T-cell antigen coupler (TAC) receptor.

In some embodiments according to any one of the modified immune cells described above, the first nucleic acid sequence and the second nucleic acid sequence are operably linked to the same promoter. In some embodiments, the first nucleic acid sequence and the second nucleic acid sequence are operably linked to separate promoters.

One aspect of the present application provides a method of producing a modified immune cell, comprising: introducing into a precursor immune cell a first nucleic acid sequence encoding a flagellin polypeptide comprising a flagellin protein or a fragment thereof, wherein the flagellin polypeptide upon expression is capable of binding to a toll-like receptor. In some embodiments, the flagellin polypeptide comprises Motif N of a flagellin protein. In some embodiments, the flagellin polypeptide comprises Motif C of a flagellin protein. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises all or a portion of an amino acid sequence having at least about 85% sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2 or SEQ ID NO: 3. In some embodiments, the flagellin polypeptide comprises all or a portion of the amino acid sequence of SEQ ID NO: 1.

In some embodiments according to any one of the methods of production described above, the flagellin polypeptide comprises an amino acid sequence having at least about 85% sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the flagellin polypeptide comprises an amino acid sequence having at least about 85% sequence identity to SEQ ID NO: 24. In some embodiments, the flagellin polypeptide comprises the amino acid sequence of SEQ ID NO: 24. In some embodiments, the flagellin polypeptide comprises an amino acid sequence having at least about 85% sequence identity to SEQ ID NO: 32. In some embodiments, the flagellin polypeptide comprises the amino acid sequence of SEQ ID NO: 32.

In some embodiments according to any one of the methods of production described above, the flagellin polypeptide is membrane-bound. In some embodiments, the flagellin polypeptide is bound to the cell membrane via a glycosylphosphatidylinositol (GPI) linker. In some embodiments, the flagellin polypeptide is bound to the cell membrane via a transmembrane domain. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge region, such as a CD8 hinge region. In some embodiments, the flagellin polypeptide further comprises an intracellular signaling domain. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, Ligands of CD83 and combinations thereof. In some embodiments, the flagellin polypeptide is secreted.

In some embodiments according to any one of the methods of production described above, the precursor immune cell is selected from the group consisting of a cytotoxic T cell, a helper T cell, a natural killer (NK) cell, an NK-T cell, an iNK-T cell, an NK-T like cell, an αβT cell and a γδT cell. In some embodiments, the precursor immune cell is a cytotoxic T cell. In some embodiments, the modified immune cell is a γδT cell. In some embodiments, the precursor immune cell is a tumor-infiltrating T cell or DC-activated T cell.

In some embodiments according to any one of the methods of production described above, the precursor immune cell comprises an engineered receptor. In some embodiments, the method further comprises introducing into the precursor immune cell a second nucleic acid encoding an engineered receptor. In some embodiments, the engineered receptor is a chimeric antigen receptor (CAR). In some embodiments, the CAR is an anti-BCMA CAR. In some embodiments, the engineered receptor is a modified T-cell receptor (TCR). In some embodiments, the engineered receptor is a T-cell antigen coupler (TAC) receptor.

In some embodiments according to any one of the methods of production described above, the first nucleic acid sequence and the second nucleic acid sequence are operably linked to the same promoter. In some embodiments, the first nucleic acid sequence and the second nucleic acid sequence are operably linked to separate promoters. In some embodiments, the first nucleic acid and the second nucleic acid are on the same vector. In some embodiments, the first nucleic acid and the second nucleic acid are on separate vectors. In some embodiments, the vector is a viral vector. In some embodiments, the viral vector is selected from the group consisting of an adenoviral vector, an adeno-associated virus vector, a retroviral vector, a lentiviral vector, a herpes simplex viral vector, and derivatives thereof. In some embodiments, the vector is a non-viral vector. In some embodiments, the vector is an episomal expression vector.

In some embodiments according to any one of the methods of production described above, the method further comprises isolating or enriching immune cells comprising the first nucleic acid sequence and/or the second nucleic acid sequence.

In some embodiments according to any one of the methods of production described above, the method further comprises formulating the modified immune cells with at least one pharmaceutically acceptable carrier.

Also provided is a modified immune cell produced by the method according to any one of the methods of production described above.

Further provided is a pharmaceutical composition comprising the modified immune cell according to any one of the modified immune cells described above, and a pharmaceutically acceptable carrier.

Another aspect of the present application provides a method of treating a cancer in an individual, comprising administering to the individual an effective amount of the pharmaceutical composition according to any one of the pharmaceutical compositions described above. In some embodiments, the cancer is solid tumor. In some embodiments, the individual is human.

Another aspect of the present application provides an engineered flagellin polypeptide comprising an amino acid sequence having at least about 85% sequence identity to an amino acid sequence selected from the group consisting of SEQ ID NOs: 12, 14-16, 20, 22-24, and 28-32. In some embodiments, the engineered flagellin polypeptide comprises an amino acid sequence selected from the group consisting of SEQ ID NOs: 12, 14-16, 20, 22-24, and 28-32. In some embodiments, the engineered flagellin polypeptide comprises the amino acid sequence of SEQ ID NO: 24. In some embodiments, the flagellin polypeptide comprises the amino acid sequence of SEQ ID NO: 32.

Compositions, uses, kits and articles of manufacture comprising any one of the modified immune cells are also provided.

### BRIEF DESCRIPTION OF THE DRAWINGS

FIG. 1 shows exemplary amino acid sequences and alignment of flagellin proteins from E. coli, S. typhimurium, and S. muenchen. The italic sequences at the N-terminus are the N-terminal domain, and italic sequences at the C-terminus are the C-terminal domain. The bolded and italic sequences at the N-terminus are Motif N, and the bolded sequences at the C-terminus are Motif C. The non-italic sequences belong to the intervening hypervariable domain.

FIG. 2A illustrates design of flagellin fragments via progressive truncations at the N- and/or C-terminal regions with a GAAG linker (SEQ ID NO: 36) in place of the hypervariable regions of a full length flagellin from S. typhimurium. FIGS. 2B-2D show screening results of exemplary flagellin fragments of FIG. 2A. FIG. 2E shows a summary of biologically active flagellin fragments identified in the screens of FIGS. 2B-2D.

FIG. 3A shows design of anti-BCMA CARs armored with soluble full-length flagellin or its fragment Flic-16a. FIGS. 3B-3C show cytotoxic effects of armored anti-BCMA CARs of FIG. 3A against BCMA-positive target cells, H929, when expressed on αβ (FIG. 3B) or γδ (FIG. 3C) T cells. T cells expressing anti-BCMA CAR alone (i.e., “unarmored”) and untransduced T cells served as controls in the experiments.

FIGS. 4A-4F show cytokine release profiles of T cells expressing anti-BCMA CARs armored with full-length flagellin or its fragment Flic-16a when incubated with BCMA-positive H929 cells. In FIGS. 4A-4C, the anti-BCMA CARs were expressed on αβ T cells. In FIGS. 4D-4F, the anti-BCMA CARs were expressed on γδ T cells. T cells expressing anti-BCMA CAR alone (i.e., “unarmored”) and untransduced T cells served as controls in the experiments. FIGS. 4A and 4D show release of TNF-α. FIGS. 4B and 4E show release of IFN-γ. FIGS. 4C and 4F show release of IL-2.

### DETAILED DESCRIPTION OF THE INVENTION

The present invention provides modified immune cells expressing a flagellin polypeptide comprising a full-length flagellin protein or a fragment thereof, and methods of treating cancer using the modified immune cells. The flagellin polypeptide is capable of binding to a toll-like receptor, such as TLR5. The flagellin polypeptide may be a transmembrane molecule, or secreted from the modified immune cell. The modified immune cells described herein have potent tumor lytic activity and elicits antigen-specific T-cell response against tumor sites.

Accordingly, one aspect of the present invention provides a modified immune cell (e.g., T cell) comprising a heterologous nucleic acid sequence encoding a flagellin polypeptide comprising a flagellin protein or a fragment thereof, wherein the flagellin polypeptide upon expression is capable of binding to a toll-like receptor. In some embodiments, the flagellin polypeptide is secreted by the modified immune cell. In some embodiments, the flagellin polypeptide is bound to the cell membrane of the modified immune cell via a GPI linker. In some embodiments, the flagellin polypeptide comprises a transmembrane domain and an intracellular signaling domain derived from a co-stimulatory molecule. In some embodiments, the modified immune cell further comprises an engineered receptor, such as a chimeric antigen receptor, a modified T-cell receptor, or a T-cell antigen coupler (TAC) receptor.

Also provided are compositions (such as pharmaceutical compositions), kits and articles of manufacture comprising the modified immune cells, and methods of treating cancer using the modified immune cells described herein.

### Definitions

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of cancer means to defer, hinder, slow, retard, stabilize, and/or postpone development of the disease. This delay can be of varying lengths of time, depending on the history of the disease and/or individual being treated. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “effective amount” used herein refers to an amount of an agent or a combination of agents, sufficient to treat a specified disorder, condition or disease such as to ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other undesired cell proliferation. In some embodiments, an effective amount is an amount sufficient to delay disease development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

As used herein, an “individual” or a “subject” refers to a mammal, including, but not limited to, human, bovine, horse, feline, canine, rodent, or primate. In some embodiments, the individual is a human.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which a heterologous nucleic acid is transferred or introduced into the host cell. A “transfected” or “transformed” or “transduced” cell is one which has been transfected, transformed or transduced with a heterologous nucleic acid. The cell includes the primary subject cell and its progeny.

“Adjuvant setting” refers to a clinical setting in which an individual has had a history of cancer, and generally (but not necessarily) been responsive to therapy, which includes, but is not limited to, surgery (e.g., surgery resection), radiotherapy, and chemotherapy. However, because of their history of cancer, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

“Percent (%) amino acid sequence identity” or “homology” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software.

Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Chimeric antigen receptor” or “CAR” as used herein refers to genetically engineered receptors, which graft one or more antigen specificity onto cells, such as T cells. CARs are also known as “artificial T-cell receptors,” “chimeric T-cell receptors,” or “chimeric immune receptors.” In some embodiments, the CAR comprises an extracellular variable domain of an antibody specific for a tumor antigen, and an intracellular signaling domain of a T cell or other receptors, such as one or more co-stimulatory domains. “CAR-T” refers to a T cell that expresses a CAR

“T-cell receptor” or “TCR” as used herein refers to an endogenous or modified T-cell receptor comprising an extracellular antigen binding domain that binds to a specific antigenic peptide bound in an MHC molecule. In some embodiments, the TCR comprises a TCRα polypeptide chain and a TCR polypeptide chain. In some embodiments, the TCR comprises a TCRγ polypeptide chain and a TCR 8 polypeptide chain. In some embodiments, the TCR specifically binds a tumor antigen. “TCR-T” refers to a T cell that expresses a recombinant TCR.

“T-cell antigen coupler receptor” or “TAC receptor” as used herein refers to an engineered receptor comprising an extracellular antigen binding domain that binds to a specific antigen and a T-cell receptor (TCR) binding domain, a transmembrane domain, and an intracellular domain of a co-receptor molecule. The TAC receptor co-opts the endogenous TCR of a T cell that expressed the TAC receptor to elicit antigen-specific T-cell response against a target cell.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The term antibody includes, but is not limited to, fragments that are capable of binding antigen, such as Fv, single-chain Fv (scFv), Fab, Fab′, and (Fab′)₂. The term antibody includes conventional four-chain antibodies, and single-domain antibodies, such as heavy-chain only antibodies or fragments thereof, e.g., V_{H}H.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “cell” includes the primary subject cell and its progeny.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “an,” and “the” include plural referents unless the context clearly dictates otherwise.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the modified immune cells and methods of treatment described herein are specifically embraced by the present invention and are disclosed herein just as if each and every combination was individually and explicitly disclosed. In addition, all subcombinations of the modified immune cells listed in the embodiments describing such variables are also specifically embraced by the present invention and are disclosed herein just as if each and every such sub-combination of proteins was individually and explicitly disclosed herein.

### II. Modified Immune Cells

One aspect of the present invention provides a modified immune cell comprising a heterologous nucleic acid sequence encoding a flagellin polypeptide comprising flagellin or a fragment thereof, wherein the flagellin polypeptide upon expression is capable of binding to a toll-like receptor. In some embodiments, the toll-like receptor is selected from the group consisting of TLR4, TLR5, TLR11, TLR2, TLR5 and TLR5. In some embodiments, the flagellin polypeptide is secreted. In some embodiments, the flagellin polypeptide is membrane bound. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-T cell, an iNK-T cell, an NK-T like cell, an σβT cell, a γδT cell, a tumor-infiltrating T cell and a dendritic cell (DC)-activated T cell.

In some embodiments, there is provided a modified immune cell comprising a heterologous nucleic acid sequence encoding a flagellin polypeptide comprising a flagellin protein or a fragment thereof, wherein the flagellin polypeptide is capable of binding to TLR5. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding a secreted flagellin polypeptide comprising a flagellin protein or a fragment thereof, wherein the flagellin polypeptide is capable of binding to a toll-like receptor (e.g., TLR5). In some embodiments, the flagellin polypeptide consists of or consists essentially of a flagellin protein or a fragment thereof. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding a flagellin polypeptide comprising a flagellin protein or a fragment thereof and a GPI-anchoring peptide sequence, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the GPI-anchoring peptide sequence is attached to a GPI linker. In some embodiments, the GPI-anchoring peptide sequence is located at the C-terminus of the flagellin polypeptide. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding a flagellin polypeptide comprising a flagellin protein or a fragment thereof and a transmembrane domain, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a heterologous nucleic acid sequence encoding a flagellin polypeptide comprising a flagellin protein or a fragment thereof, a transmembrane domain and an intracellular signaling domain, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, Ligands of CD83 and combinations thereof. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a first heterologous nucleic acid sequence encoding a flagellin polypeptide comprising a flagellin protein or a fragment thereof, wherein the flagellin polypeptide is capable of binding to TLR5; and a second heterologous nucleic acid sequence encoding an engineered receptor. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the engineered receptor is a CAR, such as an anti-BCMA CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a first heterologous nucleic acid sequence encoding a secreted flagellin polypeptide comprising a flagellin protein or a fragment thereof, wherein the flagellin polypeptide is capable of binding to a toll-like receptor (e.g., TLR5); and a second heterologous nucleic acid sequence encoding an engineered receptor. In some embodiments, the flagellin polypeptide consists of or consists essentially of a flagellin protein or a fragment thereof. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the engineered receptor is a CAR, such as an anti-BCMA CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a first heterologous nucleic acid sequence encoding a flagellin polypeptide comprising a flagellin protein or a fragment thereof and a GPI-anchoring peptide sequence, wherein the flagellin polypeptide is capable of binding to a toll-like receptor (e.g., TLR5); and a second heterologous nucleic acid sequence encoding an engineered receptor. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the GPI-anchoring peptide sequence is attached to a GPI linker. In some embodiments, the GPI-anchoring peptide sequence is located at the C-terminus of the flagellin polypeptide. In some embodiments, the engineered receptor is a CAR, such as an anti-BCMA CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a first heterologous nucleic acid sequence encoding a flagellin polypeptide comprising a flagellin protein or a fragment thereof and a transmembrane domain, wherein the flagellin polypeptide is capable of binding to a toll-like receptor (e.g., TLR5); and a second heterologous nucleic acid sequence encoding an engineered receptor. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8. In some embodiments, the engineered receptor is a CAR, such as an anti-BCMA CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a modified immune cell comprising a first heterologous nucleic acid sequence encoding a flagellin polypeptide comprising a flagellin protein or a fragment thereof, a transmembrane domain and an intracellular signaling domain, wherein the flagellin polypeptide is capable of binding to a toll-like receptor (e.g., TLR5); and a second heterologous nucleic acid sequence encoding an engineered receptor. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, Ligands of CD83 and combinations thereof. In some embodiments, the engineered receptor is a CAR, such as an anti-BCMA CAR. In some embodiments, the engineered receptor is an engineered TCR. In some embodiments, the engineered receptor is a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a CAR-expressing immune cell (e.g., CAR-T cell) comprising a heterologous nucleic acid sequence encoding a flagellin polypeptide comprising flagellin or a fragment thereof, wherein the flagellin polypeptide upon expression is capable of binding to a toll-like receptor (e.g., TLR5). In some embodiments, the toll-like receptor is selected from the group consisting of TLR4, TLR5, TLR11, TLR2, TLR3 and TLR9. In some embodiments, the flagellin polypeptide is secreted. In some embodiments, the flagellin polypeptide consists of or consists essentially of a flagellin protein or a fragment thereof. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the flagellin polypeptide is membrane bound. In some embodiments, the flagellin polypeptide comprises a GPI-anchoring peptide sequence. In some embodiments, the GPI-anchoring peptide sequence is attached to a GPI linker. In some embodiments, the GPI-anchoring peptide sequence is located at the C-terminus of the flagellin polypeptide. In some embodiments, the flagellin polypeptide comprises a transmembrane domain. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8. In some embodiments, the flagellin polypeptide further comprises an intracellular signaling domain. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, Ligands of CD83 and combinations thereof. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a TCR-expressing immune cell (e.g., TCR-T cell) comprising a heterologous nucleic acid sequence encoding a flagellin polypeptide comprising flagellin or a fragment thereof, wherein the flagellin polypeptide upon expression is capable of binding to a toll-like receptor (e.g., TLR5). In some embodiments, the toll-like receptor is selected from the group consisting of TLR4, TLR5, TLR11, TLR2, TLR3 and TLR9. In some embodiments, the flagellin polypeptide is secreted. In some embodiments, the flagellin polypeptide consists of or consists essentially of a flagellin protein or a fragment thereof. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the flagellin polypeptide is membrane bound. In some embodiments, the flagellin polypeptide comprises a GPI-anchoring peptide sequence. In some embodiments, the GPI-anchoring peptide sequence is attached to a GPI linker. In some embodiments, the GPI-anchoring peptide sequence is located at the C-terminus of the flagellin polypeptide. In some embodiments, the flagellin polypeptide comprises a transmembrane domain. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8. In some embodiments, the flagellin polypeptide further comprises an intracellular signaling domain. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, Ligands of CD83 and combinations thereof. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a TAC-expressing immune cell (e.g., TAC-T cell) comprising a heterologous nucleic acid sequence encoding a flagellin polypeptide comprising flagellin or a fragment thereof, wherein the flagellin polypeptide upon expression is capable of binding to a toll-like receptor (e.g., TLR5). In some embodiments, the toll-like receptor is selected from the group consisting of TLR4, TLR5, TLR11, TLR2, TLR3 and TLR9. In some embodiments, the flagellin polypeptide is secreted. In some embodiments, the flagellin polypeptide consists of or consists essentially of a flagellin protein or a fragment thereof. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the flagellin polypeptide is membrane bound. In some embodiments, the flagellin polypeptide comprises a GPI-anchoring peptide sequence. In some embodiments, the GPI-anchoring peptide sequence is attached to a GPI linker. In some embodiments, the GPI-anchoring peptide sequence is located at the C-terminus of the flagellin polypeptide. In some embodiments, the flagellin polypeptide comprises a transmembrane domain. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8. In some embodiments, the flagellin polypeptide further comprises an intracellular signaling domain. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, Ligands of CD83 and combinations thereof. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

In some embodiments, there is provided a CAR-expressing immune cell (e.g., CAR-T cell) comprising a heterologous nucleic acid sequence encoding a flagellin polypeptide comprising an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 24. In some embodiments, there is provided a CAR-expressing immune cell (e.g., CAR-T cell) comprising a heterologous nucleic acid sequence encoding a flagellin polypeptide comprising the amino acid sequence of SEQ ID NO: 24. In some embodiments, there is provided a CAR-expressing immune cell (e.g., CAR-T cell) comprising a heterologous nucleic acid sequence encoding a flagellin polypeptide comprising an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 32. In some embodiments, there is provided a CAR-expressing immune cell (e.g., CAR-T cell) comprising a heterologous nucleic acid sequence encoding a flagellin polypeptide comprising the amino acid sequence of SEQ ID NO: 32. In some embodiments, there is provided a CAR-expressing immune cell (e.g., CAR-T cell) comprising a heterologous nucleic acid sequence encoding a flagellin polypeptide comprising an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1. In some embodiments, there is provided a CAR-expressing immune cell (e.g., CAR-T cell) comprising a heterologous nucleic acid sequence encoding a flagellin polypeptide comprising the amino acid sequence of SEQ ID NO: 1. In some embodiments, the CAR is an anti-BCMA CAR. In some embodiments, the immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell.

### Immune Cells

The modified immune cell can be derived from a variety of cell types and cell sources. Cells from any mammalian species, including, but not limited to, mice, rats, guinea pigs, rabbits, dogs, monkeys, and humans, are contemplated herein. In some embodiments, the modified immune cell is a human cell. In some embodiments, the modified immune cell is allogenic (i.e., from the same species, but different donor) as the recipient individual. In some embodiments, the modified immune cell is autologous (i.e., the donor and the recipient are the same). In some embodiments, the modified immune cell is syngeneic (i.e., the donor and the recipients are different individuals, but are identical twins).

In some embodiments, the modified immune cell is derived from a primary cell. In some embodiments, the modified immune cell is a primary cell isolated from an individual. In some embodiments, the modified immune cell is propagated (such as proliferated and/or differentiated) from a primary cell isolated from an individual. In some embodiments, the primary cell is of the hematopoietic lineage. In some embodiments, the primary cell is obtained from the thymus. In some embodiments, the primary cell is obtained from the lymph or lymph nodes (such as tumor draining lymph nodes). In some embodiments, the primary cell is obtained from the spleen. In some embodiments, the primary cell is obtained from the bone marrow. In some embodiments, the primary cell is obtained from the blood, such as the peripheral blood. In some embodiments, the primary cell is a Peripheral Blood Mononuclear Cell (PBMC). In some embodiments, the primary cell is derived from the blood plasma. In some embodiments, the primary cell is derived from a tumor. In some embodiments, the primary cell is obtained from the mucosal immune system. In some embodiments, the primary cell is obtained from a biopsy sample.

In some embodiments, the modified immune cell is derived from a cell line. In some embodiments, the modified immune cell is obtained from a commercial cell line. In some embodiments, the modified immune cell is a cell line established from a primary cell isolated from an individual. In some embodiments, the modified immune cell is propagated (such as proliferated and/or differentiated) from a cell line. In some embodiments, the cell line is mortal. In some embodiments, the cell line is immortalized. In some embodiments, the cell line is a tumor cell line, such as a leukemia or lymphoma cell line. In some embodiments, the cell line is a cell line derived from the PBMC. In some embodiments, the cell line is a stem cell line. In some embodiments, the cell line is selected from the group consisting of HEK293-6E cells, NK-92 cells, and Jurkat cells.

Exemplary immune cells useful for the present invention include, but are not limited to, dendritic cells (including immature dendritic cells and mature dendritic cells), T lymphocytes (such as naïve T cells, effector T cells, memory T cells, cytotoxic T lymphocytes, T helper cells, Natural Killer T cells, Treg cells, tumor infiltrating lymphocytes (TIL), and lyphokine-activated killer (LAK) cells), B cells, Natural Killer (NK) cells, monocytes, macrophages, neutrophils, granulocytes, and combinations thereof. Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In the cases that the pharmaceutical composition comprises a plurality of modified immune cells, the modified immune cells can be a specific subpopulation of an immune cell type, a combination of subpopulations of an immune cell type, or a combination of two or more immune cell types. In some embodiments, the immune cell is present in a homogenous cell population. In some embodiments, the immune cell is present in a heterogeneous cell population that is enhanced in the immune cell. In some embodiments, the modified immune cell is a lymphocyte. In some embodiments, the modified immune cell is not a lymphocyte. In some embodiments, the modified immune cell is suitable for adoptive immunotherapy. In some embodiments, the modified immune cell is a PBMC. In some embodiments, the modified immune cell is an immune cell derived from the PBMC. In some embodiments, the modified immune cell is a T cell. In some embodiments, the modified immune cell is a CD4⁺ T cell. In some embodiments, the modified immune cell is a CD8⁺ T cell. In some embodiments, the modified immune cell is a B cell. In some embodiments, the modified immune cell is an NK cell.

In some embodiments, the modified immune cell is derived from a stem cell. In some embodiments, the stem cell is a totipotent stem cell. In some embodiments, the stem cell is a pluripotent stem cell. In some embodiments, the stem cell is a unipotent stem cell. In some embodiments, the stem cell is a progenitor cell. In some embodiments, the stem cell is an embryonic stem cell. In some embodiments, the stem cell is hematopoietic stem cell. In some embodiments, the stem cell is a mesenchymal stem cell. In some embodiments, the stem cell is an induced pluripotent stem cell (iPSC).

The modified immune cell may comprise any number (such as any of 1, 2, 3, 4, 5, 10, 50, 100, 1000, or more) of the heterologous nucleic acid sequence (including first and second nucleic acid sequences). In some embodiments, the modified immune cell comprises a single copy of the first and/or second heterologous nucleic acid sequence. In some embodiments, the modified immune cell comprises a plurality of copies of the first and/or second heterologous nucleic acid sequence. In some embodiments, the modified immune cell further comprises at least one additional heterologous nucleic acid sequence, for example, a heterologous nucleic acid sequence encoding an immunomodulatory agent, such as cytokine, chemokine, and/or an immune checkpoint inhibitor.

Nucleic acid(s) comprising the heterologous nucleic acid sequence(s) described herein may be transiently or stably incorporated in the modified immune cell. In some embodiments, the nucleic acid(s) is transiently expressed in the modified immune cell. For example, the nucleic acid(s) may be present in the nucleus of the modified immune cell in an extrachromosomal array. The nucleic acid(s) may be introduced into the modified immune cell using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection.

In some embodiments, the heterologous nucleic acid sequence(s) is present in the genome of the modified immune cell. For example, nucleic acid(s) comprising the heterologous nucleic acid sequence(s) may be integrated into the genome of the modified immune cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN®), CRISPR/Cas9, and zinc-finger nucleases. In some embodiments, the heterologous nucleic acid sequence(s) is integrated in a specifically designed locus of the genome of the modified immune cell. In some embodiments, the heterologous nucleic acid sequence(s) is integrated in an integration hotspot of the genome of the modified immune cell. In some embodiments, the heterologous nucleic acid (sequence) is integrated in a random locus of the genome of the modified immune cell. In the cases that multiple copies of the heterologous nucleic acid sequence(s) are present in a single modified immune cell, the heterologous nucleic acid sequences may be integrated in a plurality of loci of the genome of the modified immune cell.

### Flagellin Polypeptide

The modified immune cells described herein express a flagellin polypeptide capable of binding to a toll-like receptor (TLR). The present application also provides flagellin polypeptides and compositions thereof.

In some embodiments, there is provided a flagellin polypeptide comprising a flagellin protein or a fragment thereof, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32.

In some embodiments, there is provided a secreted flagellin polypeptide comprising a flagellin protein or a fragment thereof, wherein the flagellin polypeptide is capable of binding to a toll-like receptor (e.g., TLR5). In some embodiments, the flagellin polypeptide consists of or consists essentially of a flagellin protein or a fragment thereof. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32.

In some embodiments, there is provided a flagellin polypeptide comprising a flagellin protein or a fragment thereof and a GPI-anchoring peptide sequence, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the GPI-anchoring peptide sequence is attached to a GPI linker. In some embodiments, the GPI-anchoring peptide sequence is located at the C-terminus of the flagellin polypeptide.

In some embodiments, there is provided a flagellin polypeptide comprising a flagellin protein or a fragment thereof and a GPI linker, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32.

In some embodiments, there is provided a flagellin polypeptide comprising a flagellin protein or a fragment thereof and a transmembrane domain, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8.

In some embodiments, there is provided a flagellin polypeptide comprising a flagellin protein or a fragment thereof, a transmembrane domain and an intracellular signaling domain, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8.

In some embodiments, there is provided a flagellin polypeptide comprising a flagellin protein or a fragment thereof, a transmembrane domain and a co-stimulatory signaling domain, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, Ligands of CD83 and combinations thereof.

In some embodiments, the flagellin polypeptide is capable of binding to a TLR selected from the group consisting of TLR4, TLR5, TLR11, TLR2, TLR3, and TLR9. In some embodiments, the flagellin polypeptide is capable of binding to TLR5. In some embodiments, the flagellin polypeptide is capable of binding to TLR5 homodimer. In some embodiments, the flagellin polypeptide is capable of binding to TLR4/TLR5 heterodimer. In some embodiments, the flagellin polypeptide is capable of binding to TLR11. In some embodiments, the flagellin polypeptide is capable of binding to more than one TLR. In some embodiments, the flagellin polypeptide is capable of binding to both TLR5 homodimer and TLR4/TLR5 heterodimer. In some embodiments, the flagellin polypeptide is capable of binding to TLR5 and TLR11. In some embodiments, the binding affinity of the flagellin polypeptide to the TLR is about 10⁻¹⁰ M to about 10⁻³ M, such as about 10⁻¹⁰ M to about 10⁻⁸M, about 10⁻⁸ M to about 10⁻⁶ M, or 10⁻⁶M to about 10⁻³ M.

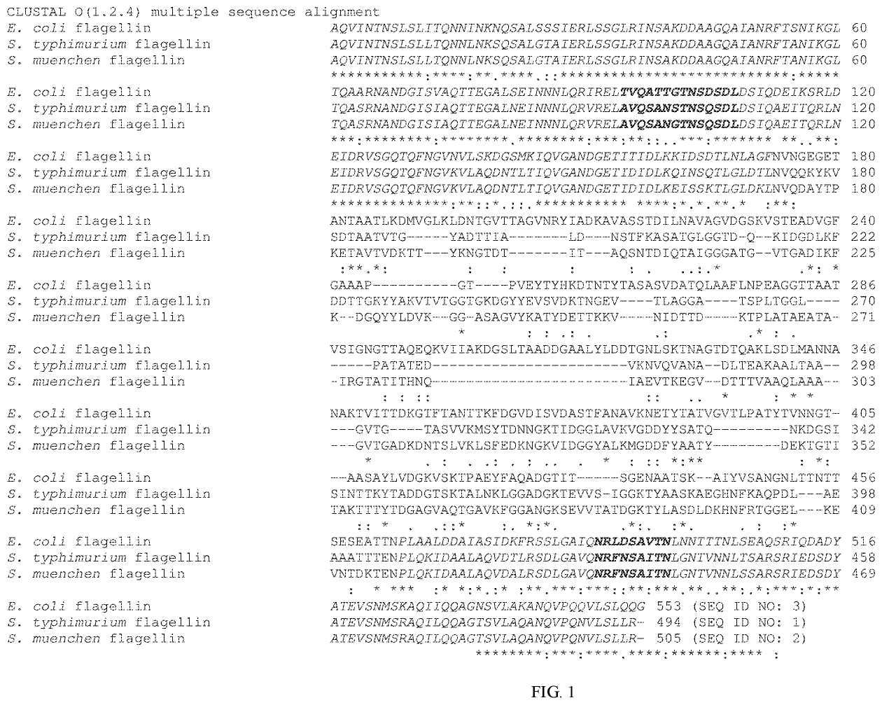

The flagellin polypeptide may be derived from any naturally occurring flagellin proteins that bind to a TLR such as TLR5 and/or elicit an immune response. Flagellin is the structural component of flagellum, a locomotory organ that is mostly associated with Gram-negative bacteria. It is characterized by highly conserved N- and C-terminal domains with an intervening hypervariable domain that have highly variable sequences and lengths across different bacterial species. Three-dimensional structure of Salmonella enterica FliC flagellin has been solved (PDB entry IUCU), which shows that the N-terminal and C-terminal domains of flagellin form the coiled-coil domains D0 and D1, and the intervening hypervariable domain D2 and D3 consists mostly of β-strands. The N-terminal helical bundle in D1 is followed by two β-turns and a β-hairpin. See, Samatey F. A. et al. Nature (2001) 410: 331-337. Based on alignments of 202 flagellin sequences, it is found that all flagellins contain a conserved block of about 140 residues from the start codon at the N-terminus, corresponding to the ND0, ND1a and ND1b subdomains and the β-turn. Also, all flagellins contain a conserved block of about 90 residues at the C-terminus, corresponding to CD1 and CD0 helices. See, Beatson S A et al. (2006) TRENDS in Microbiology 14(4): 151-155. Mutagenesis studies have been carried out on Salmonella muenchen flagellin (SEQ ID NO: 2), which identify a “Motif N” and a “Motif C” that are required for pro-inflammatory signaling by flagellin. Motif N corresponds to amino acid residues 95-108 in the N-terminus of S. muenchen flagellin, and Motif C corresponds to amino acid residues 441-449 in the C-terminus of S. muenchen flagellin, wherein the amino acid numbering is based on SEQ ID NO: 2. See, Murthy K G K et al. J. Biol. Chem. (2004) 279(7): 5667-5675; and Donnelly M A and Steiner T S. J. Biol. Chem. (2002) 277(43): 40456-40461. Alignment of three exemplary bacterial flagellin sequences (SEQ ID NOs: 1-3) and the corresponding domains and Motifs are shown in FIG. 1.

In some embodiments, the flagellin polypeptide is derived from a flagellin protein of a Gram negative bacterium. In some embodiments, the flagellin polypeptide is derived from a flagellin protein of a Gram positive bacterium. In some embodiments, the flagellin polypeptide is derived from a flagellin protein of a bacterium of a species selected from the group consisting of Serratia, Proteus, Pseudomonas, Escherichia, Listeria, Salmonella, Vibrio, and Yersinia. In some embodiments, the flagellin polypeptide is derived from a Salmonella species, such as S. typhimurium or S. muenchen. In some embodiments, the flagellin polypeptide is derived from E. coli. In some embodiments, the flagellin polypeptide is derived from a Vibrio species, such as Vibrio vulnificus, e.g., FlaB of V. vulnificus. See, Lee S E et al., Infection and Immunity, 74(1):694-702 (2006); Zheng J H et al., Sci. Trans. Med. 9: eaak9537 (2017).

In some embodiments, the flagellin polypeptide comprises a full-length flagellin protein. In some embodiments, the flagellin polypeptide comprises an amino acid sequence selected from the group consisting of SEQ ID NOs: 1-3 and 8. Full-length flagellin protein sequences are known in the art, including, but not limited to sequences with UniProt (worldwide web.uniprot.org) access numbers Q9ZBA2, Q7MMU2, Q7MIM4, Q56702, Q9KQ61, Q56574, Q56571, 034221, Q7MIM5, Q56703, Q56572, 034223, Q6R4Q0, Q6R4P9, Q5E3N9, Q5E322, Q9KQ60, Q56570, Q7MIM3, 087081, Q6R4Q1, Q60246, Q58FG3, Q7MMU6, Q5E3N5, Q6LTQ2, Q6LTQ1, Q8ECA6, Q8ECA5, Q9R9R9, Q5MBN1, Q5MBN0, 030377, 030376, Q5QZT0, Q884Y7, Q76M63, Q8VS56, Q52079, P21990, P21991, P21989, Q73NZ6, Q9KWX0, Q9KWW9, Q73MN3, P21992, Q9ANU7, Q72S55, P80160, Q5XPI2, Q26501, Q9WZL7, Q8RCD3, Q9KCU2, P02968, Q65EB8, Q5WBM8, Q8EMW3, 069136, Q8RRA1, Q893U2, Q8RR97, Q5KV70, Q8RRA0, Q05203, Q5KV59, Q67K41, Q6QA53, Q52694, 031059, Q6QA52, Q9S526, Q9RQU7, Q9FA23, Q9S0T2, Q93ES1, Q842B7, Q9L9M2, Q83XM5, Q842D2, Q6VMU0, Q9L9M0, Q842D5, Q93ES3, Q842B4, Q9L9M1, Q842A8, Q6VMT9, Q6VMU2, Q5PEW3, Q53834, Q6V2P2, Q75SX0, Q7N5J4, Q56826, Q54444, P13713, Q8GNT8, Q75SX7, Q75SY3, Q75SW4, Q74UY9, Q66PN8, P42273, P42272, Q76DK5, Q6V2M6, Q54864, Q6V2U0, Q56912, Q5DW30, Q8RST5, Q8D3D7, 033578, Q81SF2, Q63D82, Q73AJ3, Q6HKP2, Q81FD5, Q79AJ2, Q79AJ1, Q73AJ4, Q6HKP3, Q81FD6, Q5Y833, Q6LW29, Q93TL9, Q93TL8, Q9AET1, Q03473, Q7NTP3, Q5DY03, Q8CZT1, Q9XB38, Q6VYQ2, Q7TTM9, Q07911, Q07910, Q7VF81, Q9XB37, Q7M7N1, Q56746, Q9R954, Q6L5K3, Q6L5KO, P96751, Q93GT1, P46210, 067803, Q89NY8, Q6NC33, Q9REF9, Q9F4K8, Q9F4K7, P96307, P96309, P96308, Q03842, P58330, P13119, P13118, Q6QMR9, 034166, Q52943, Q89F36, Q89F35, Q98HD0, Q98HC9, 052068, Q43896, P18914, Q5LMV3, Q5FST5, Q6AGB4, Q9KGT9, Q6AMN5, Q6AJQ8, Q748G4, Q6H8R2, Q6MIU6, Q6MI33, Q6MQ75, Q6MQ77, Q729A8, Q72AB5, Q72C43, Q9FAE7, Q8P9C4, Q5GZN6, Q8PL31, Q82UA3, Q48824, Q5X5M6, P53606, Q7NRA5, Q7NRA4, Q9Z3A8, 068144, Q9S639, and Q76BR3.

In some embodiments, the flagellin polypeptide comprises a portion of a naturally occurring flagellin protein that is capable of binding to the TLR. In some embodiments, the flagellin polypeptide comprises Motif N of a naturally occurring flagellin protein, which corresponds to amino acids 95-108 of SEQ ID NO: 2. In some embodiments, the flagellin polypeptide comprises Motif C of a naturally occurring flagellin protein, which corresponds to amino acids 441-449 of SEQ ID NO: 2. In some embodiments, the flagellin polypeptide comprises both Motif N and Motif C.

In some embodiments, the flagellin protein or fragment thereof comprises at least about any one of 50, 60, 70, 80, 90, 100, 125, 150, 175, 200, 250, 300, 350, 400, 450, 500, 550, 600 or more amino acids. In some embodiments, the flagellin protein or fragment thereof comprises no more than about any one of 600, 550, 500, 450, 350, 300, 250, 200, 175, 150, 125, 100, 90, 80, 70, 60, 50, or fewer amino acids. In some embodiments, the flagellin protein or fragment thereof comprises about any one of 50-60, 50-75, 50-100, 50-150, 50-200, 50-250, 100-150, 100-200, 100-250, 150-250, 250-500, or 50-550 amino acids.

In some embodiments, the flagellin polypeptide comprises the N-terminal domain of a naturally occurring flagellin protein, corresponding to amino acids 1-172 of SEQ ID NO: 2. In some embodiments, the flagellin polypeptide comprises amino acids 1-172 of SEQ ID NO: 1. In some embodiments, the flagellin polypeptide comprises amino acids 1-172 of SEQ ID NO: 2. In some embodiments, the flagellin polypeptide comprises amino acids 1-172 of SEQ ID NO: 3. In some embodiments, the flagellin polypeptide comprises any one of the N-terminal domains as described in Beatson S A et al. (2006) TRENDS in Microbiology 14(4): 151-155. In some embodiments, the flagellin polypeptide comprises a truncated C-terminal domain of a naturally occurring flagellin protein, lacking no more than about any one of 95, 90, 85, 80, 75, 70, 65, 60, 55, 50, 45, 40, 35, 30, 25, 20, 15, 10, 5 or fewer amino acids at the N-terminus. In some embodiments, the flagellin polypeptide comprises a truncated N-terminal domain of a naturally occurring flagellin protein, corresponding to amino acids 53-172, 81-172, or 95-172 of SEQ ID NO: 2.

In some embodiments, the flagellin polypeptide comprises the C-terminal domain of a naturally occurring flagellin protein, corresponding to amino acids 418-505 of SEQ ID NO: 2. In some embodiments, the flagellin polypeptide comprises amino acids 407-494 of SEQ ID NO: 1. In some embodiments, the flagellin polypeptide comprises amino acids 418-505 of SEQ ID NO: 2. In some embodiments, the flagellin polypeptide comprises amino acids 465-553 of SEQ ID NO: 3. In some embodiments, the flagellin polypeptide comprises any one of the C-terminal domains as described in Beatson S A et al. (2006) TRENDS in Microbiology 14(4): 151-155. In some embodiments, the flagellin polypeptide comprises a truncated N-terminal domain of a naturally occurring flagellin protein, lacking no more than about any one of 45, 40, 35, 30, 25, 20, 15, 10, 5 or fewer amino acids at the C-terminus. In some embodiments, the flagellin polypeptide comprises a truncated C-terminal domain of a naturally occurring flagellin protein, corresponding to amino acids 450-505, 460-505, 470-505, or 481-505 of SEQ ID NO: 2.

In some embodiments, the flagellin polypeptide comprises both the N-terminal domain and the C-terminal domain of a naturally occurring flagellin protein. In some embodiments, the flagellin polypeptide comprises any one of the truncated N-terminal domain as described herein and the C-terminal domain of a naturally occurring flagellin protein. In some embodiments, the flagellin polypeptide comprises the N-terminal domain and any one of the truncated C-terminal domain as described herein of a naturally occurring flagellin protein. In some embodiments, the flagellin polypeptide comprises any one of the truncated N-terminal domain as described herein and any one of the truncated C-terminal domain as described herein of a naturally occurring flagellin protein.

In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises a truncated N-terminal domain comprising Motif N of a flagellin protein and a truncated C-terminal domain comprising Motif C of the flagellin protein, wherein the N-terminal domain and the C-terminal domain are fused to each other via a peptide linker. In some embodiments, the flagellin polypeptide comprises the N-terminal domain of any one of the amino acid sequences of SEQ ID NOs: 9-32, and/or the C-terminal domain of any one of the amino acid sequences of SEQ ID NOs: 9-32. In some embodiments, the flagellin polypeptide comprises the N-terminal domain and the C-terminal domain of any one of the amino acid sequences of SEQ ID NOs: 9-32. In some embodiments, the N-terminal domain is fused to the C-terminal domain via a peptide linker comprising the amino acid sequence of SEQ ID NO: 36 (GAAG).

In some embodiments, the flagellin polypeptide comprises the intervening hypervariable domain or a portion thereof of a naturally occurring flagellin protein. In some embodiments, the flagellin polypeptide does not comprise the intervening hypervariable domain of a naturally occurring flagellin protein. In some embodiments, the flagellin polypeptide comprises a fusion protein comprising the N-terminal domain or a truncated fragment thereof fused to the C-terminal domain or a truncated fragment thereof. In some embodiments, the flagellin polypeptide comprises a peptide linker disposed between the N-terminal domain or a truncated fragment thereof and the C-terminal domain or a truncated fragment thereof. In some embodiments, the peptide linker is derived from the intervening hypervariable domain of a naturally occurring flagellin protein. In some embodiments, the peptide linker does not correspond to any intervening hypervariable domain sequence of naturally occurring flagellin protein. In some embodiments, the peptide linker is a flexible peptide linker. In some embodiments, the peptide linker has low immunogenicity. In some embodiments, the peptide linker has a length of at least about any one of 5, 10, 15, 20, 25, 30, 40, 50, 72, 100 or more amino acids.

The flagellin polypeptide may comprise one or more peptide linkers disposed between different domains. For example, the N-terminal domain (e.g., a truncated N-terminal domain) and the C-terminal domain (e.g., a truncated C-terminal domain) can be fused to each other via a peptide bond or via a peptide linker. The peptide linkers connecting different domains may be the same or different. Each peptide linker can be optimized individually. The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50 or more amino acids long. In some embodiments, the peptide linker is no more than about any of 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, or about 1 amino acid to about 50 amino acids.

The peptide linker may have a naturally occurring sequence, or a non-naturally occurring sequence. In some embodiments, the peptide linker is a flexible linker. Exemplary flexible linkers include glycine polymers (G)n, glycine-serine polymers (including, for example, (GS)ₙ (SEQ ID NO: 37), (GSGGS)ₙ (SEQ ID NO: 38) and (GGGS)ₙ (SEQ ID NO: 39), where n is an integer of at least one), glycine-alanine polymers, alanine-serine polymers, and other flexible linkers known in the art. In some embodiments, the peptide linker has the amino acid sequence of SEQ ID NO: 36.

In some embodiments, the flagellin polypeptide comprises an amino acid sequence variant of a naturally occurring flagellin protein or a fragment (e.g., N-terminal domain or a truncated N-terminal domain, and/or C-terminal domain or a truncated C-terminal domain) thereof. For example, it may be desirable to improve the binding affinity and/or other biological properties of the flagellin polypeptide. Amino acid sequence variants of a flagellin polypeptide thereof may be prepared by introducing appropriate modifications into the nucleotide sequence encoding the flagellin polypeptide, or by peptide synthesis. Such modifications include, for example, deletions from, and/or insertions into and/or substitutions of residues within the amino acid sequences of the flagellin polypeptide. Any combination of deletion, insertion, and substitution can be made to arrive at the final construct, provided that the final construct possesses the desired characteristics, e.g., TLR-binding and/or pro-inflammatory activities. Assays for determining activities of flagellin polypeptides are known in the art, for example, see, Murthy K G K et al. J. Biol. Chem. (2004) 279(7): 5667-5675; Donnelly M A and Steiner T S. J. Biol. Chem. (2002) 277(43): 40456-40461; and Crellin N K et al. J. Immunol. (2005) 175:8051-8059.

In some embodiments, the flagellin polypeptide comprises a flagellin protein or fragment thereof having one or more (e.g., at least 1, 2, 3, 4, 5, 10, 15, 20 amino acids or more) conservative substitutions compared to the sequence of a naturally occurring flagellin protein or fragment thereof. In some embodiments, the flagellin polypeptide comprises a flagellin protein or fragment thereof having at least about 80% sequence identity, such as at least about any one of 85%, 87%, 90%, 91%, 92%, 93%, 94%, 95%, 96%, 97%, 98%, 99% or more sequence identity to the sequence of a naturally occurring flagellin protein or fragment thereof.

Conservative substitutions are shown in Table 1 below.

TABLE 1
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:

a. hydrophobic: Norleucine, Met, Ala, Val, Leu, Ile;

b. neutral hydrophilic: Cys, Ser, Thr, Asn, Gln;

c. acidic: Asp, Glu;

d. basic: His, Lys, Arg;

e. residues that influence chain orientation: Gly, Pro;

f. aromatic: Trp, Tyr, Phe.

Non-conservative substitutions will entail exchanging a member of one of these classes for another class.

One of skill in the art will recognize that any suitable method can be used for generating mutations in a gene of interest, including mutagenesis, polymerase chain reaction, homologous recombination, or any other genetic engineering technique known to a person of skill in the art. A mutation may involve a single nucleotide (such as a point mutation, which involves the removal, addition or substitution of a single nucleotide base within a DNA sequence) or it may involve the insertion or deletion of large numbers of nucleotides. Mutations can arise spontaneously as a result of events such as errors in the fidelity of DNA replication, or induced following exposure to chemical or physical mutagens. A mutation can also be site-directed through the use of particular targeting methods that are well known to persons of skill in the art.

A useful method for identification of residues or regions of a polypeptide that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the polypeptide agent with its target (e.g., flagellin variant and TLR5) is affected. Further substitutions may be introduced at the amino acid locations demonstrating functional sensitivity to the initial substitutions. Alternatively, or additionally, a crystal structure of flagellin: TLR5 complex can be determined to identify contact points between flagellin and TLR5. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include a flagellin polypeptide with an N-terminal methionyl residue. Exemplary substitution and insertion variants of E. coli flagellin that preserve pro-inflammatory properties are described in Donnelly M A and Steiner T S. J. Biol. Chem. (2002) 277(43): 40456-40461.

Exemplary engineered flagellin polypeptide sequences are shown in FIG. 1 and Table 2 below. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 24. In some embodiments, the flagellin polypeptide comprises the amino acid sequence of SEQ ID NO: 24. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 32. In some embodiments, the flagellin polypeptide comprises the amino acid sequence of SEQ ID NO: 32.

TABLE 2
Amino acid sequence (peptide linker sequence is in lowercase; N-terminal
domain is from the N-terminus to the amino acid residue immediately 5′ to
the peptide linker sequence, and C-terminal domain is from the amino acid
residue immediately 3′ to the peptide linker sequence to the C-terminus)

Salmonella typhimurium flagellin fragment Flic-1a
GISIAQTTEGALNEINNNLQRVRELAVQSANSTNSQSDLDSIQAEITQRLNEIDRVSGQTQFNGVKV
LAQDNTLTIQVGANDGETIDIDLKQINSQTLGLDgaagKIDAALAQVDTLRSDLGAVQNRFNSAITN
LGNTVNNLTSARSRIEDSDYATEVSNMSRAQI (SEQ ID NO: 9)
Salmonella typhimurium flagellin fragment Flic-2a
GISIAQTTEGALNEINNNLQRVRELAVQSANSTNSQSDLDSIQAEITQRLNEIDRVSGQTQFNGVKV
LAQDNTLTIQVGANDGETIDIDLKQINSQTLGLDTLNgaagKIDAALAQVDTLRSDLGAVQNRFNSA
ITNLGNTVNNLTSARSRIEDSDYATEVSNMSRAQI (SEQ ID NO: 10)
Salmonella typhimurium flagellin fragment Flic-3a
GISIAQTTEGALNEINNNLQRVRELAVQSANSTNSQSDLDSIQAEITQRLNEIDRVSGQTQFNGVKV
LAQDNTLTIQVGANDGETIDIDLKQINSQTLGLDgaagATTTENPLQKIDAALAQVDTLRSDLGAVQ
NRFNSAITNLGNTVNNLTSARSRIEDSDYATEVSNMSRAQI (SEQ ID NO: 11)
Salmonella typhimurium flagellin fragment Flic-4a
GISIAQTTEGALNEINNNLQRVRELAVQSANSTNSQSDLDSIQAEITQRLNEIDRVSGQTQFNGVKV
LAQDNTLTIQVGANDGETIDIDLKQINSQTLGLDgaagKIDAALAQVDTLRSDLGAVQNRFNSAITN
LGNTVNNLTSARSRIEDSDYATEVSNMSRAQILQQAGTSVLAQANQVPQNVLSLLR (SEQ ID NO:
12)
Salmonella typhimurium flagellin fragment Flic-5a
GISIAQTTEGALNEINNNLQRVRELAVQSANSTNSQSDLDSIQAEITQRLNEIDRVSGQTQFNGVKV
LAQDNTLTIQVGANDGETIDIDLKQINSQTLGLDTLNgaagATTTENPLQKIDAALAQVDTLRSDLG
AVQNRFNSAITNLGNTVNNLTSARSRIEDSDYATEVSNMSRAQI (SEQ ID NO: 13)
Salmonella typhimurium flagellin fragment Flic-6a
GISIAQTTEGALNEINNNLQRVRELAVQSANSTNSQSDLDSIQAEITQRLNEIDRVSGQTQFNGVKV
LAQDNTLTIQVGANDGETIDIDLKQINSQTLGLDTLNgaagKIDAALAQVDTLRSDLGAVQNRFNSA
ITNLGNTVNNLTSARSRIEDSDYATEVSNMSRAQILQQAGTSVLAQANQVPQNVLSLLR (SEQ ID
NO: 14)
Salmonella typhimurium flagellin fragment Flic-7a
GISIAQTTEGALNEINNNLQRVRELAVQSANSTNSQSDLDSIQAEITQRLNEIDRVSGQTQFNGVKV
LAQDNTLTIQVGANDGETIDIDLKQINSQTLGLDgaagATTTENPLQKIDAALAQVDTLRSDLGAVQ
NRFNSAITNLGNTVNNLTSARSRIEDSDYATEVSNMSRAQILQQAGTSVLAQANQVPQNVLSLLR
(SEQ ID NO: 15)
Salmonella typhimurium flagellin fragment Flic-8a
GISIAQTTEGALNEINNNLQRVRELAVQSANSTNSQSDLDSIQAEITQRLNEIDRVSGQTQFNGVKV
LAQDNTLTIQVGANDGETIDIDLKQINSQTLGLDTLNgaagATTTENPLQKIDAALAQVDTLRSDLG
AVQNRFNSAITNLGNTVNNLTSARSRIEDSDYATEVSNMSRAQILQQAGTSVLAQANQVPQNVLS
LLR (SEQ ID NO: 16)
Salmonella typhimurium flagellin fragment Flic-9a
RINSAGISIAQTTEGALNEINNNLQRVRELAVQSANSTNSQSDLDSIQAEITQRLNEIDRVSGQTQF
NGVKVLAQDNTLTIQVGANDGETIDIDLKQINSQTLGLDgaagKIDAALAQVDTLRSDLGAVQNRF
NSAITNLGNTVNNLTSARSRIEDSDYATEVSNMSRAQI (SEQ ID NO: 17)
Salmonella typhimurium flagellin fragment Flic-10a
RINSAGISIAQTTEGALNEINNNLQRVRELAVQSANSTNSQSDLDSIQAEITQRLNEIDRVSGQTQF
NGVKVLAQDNTLTIQVGANDGETIDIDLKQINSQTLGLDTLNgaagKIDAALAQVDTLRSDLGAVQ
NRFNSAITNLGNTVNNLTSARSRIEDSDYATEVSNMSRAQI (SEQ ID NO: 18)
Salmonella typhimurium flagellin fragment Flic-1 la
RINSAGISIAQTTEGALNEINNNLQRVRELAVQSANSTNSQSDLDSIQAEITQRLNEIDRVSGQTQF
NGVKVLAQDNTLTIQVGANDGETIDIDLKQINSQTLGLDgaagATTTENPLQKIDAALAQVDTLRSD
LGAVQNRFNSAITNLGNTVNNLTSARSRIEDSDYATEVSNMSRAQI (SEQ ID NO: 19)
Salmonella typhimurium flagellin fragment Flic-12a
RINSAGISIAQTTEGALNEINNNLQRVRELAVQSANSTNSQSDLDSIQAEITQRLNEIDRVSGQTQF
NGVKVLAQDNTLTIQVGANDGETIDIDLKQINSQTLGLDgaagKIDAALAQVDTLRSDLGAVQNRF
NSAITNLGNTVNNLTSARSRIEDSDYATEVSNMSRAQILQQAGTSVLAQANQVPQNVLSLLR
(SEQ ID NO: 20)
Salmonella typhimurium flagellin fragment Flic-13a
RINSAGISIAQTTEGALNEINNNLQRVRELAVQSANSTNSQSDLDSIQAEITQRLNEIDRVSGQTQF
NGVKVLAQDNTLTIQVGANDGETIDIDLKQINSQTLGLDTLNgaagATTTENPLQKIDAALAQVDTL
RSDLGAVQNRFNSAITNLGNTVNNLTSARSRIEDSDYATEVSNMSRAQI (SEQ ID NO: 21)
Salmonella typhimurium flagellin fragment Flic-14a
RINSAGISIAQTTEGALNEINNNLQRVRELAVQSANSTNSQSDLDSIQAEITQRLNEIDRVSGQTQF
NGVKVLAQDNTLTIQVGANDGETIDIDLKQINSQTLGLDTLNgaagKIDAALAQVDTLRSDLGAVQ
NRFNSAITNLGNTVNNLTSARSRIEDSDYATEVSNMSRAQILQQAGTSVLAQANQVPQNVLSLLR
(SEQ ID NO: 22)
Salmonella typhimurium flagellin fragment Flic-15a
RINSAGISIAQTTEGALNEINNNLQRVRELAVQSANSTNSQSDLDSIQAEITQRLNEIDRVSGQTQF
NGVKVLAQDNTLTIQVGANDGETIDIDLKQINSQTLGLDgaagATTTENPLQKIDAALAQVDTLRSD
LGAVQNRFNSAITNLGNTVNNLTSARSRIEDSDYATEVSNMSRAQILQQAGTSVLAQANQVPQNV
LSLLR (SEQ ID NO: 23)
Salmonella typhimurium flagellin fragment Flic-16a
RINSAGISIAQTTEGALNEINNNLQRVRELAVQSANSTNSQSDLDSIQAEITQRLNEIDRVSGQTQF
NGVKVLAQDNTLTIQVGANDGETIDIDLKQINSQTLGLDTLNgaagATTTENPLQKIDAALAQVDTL
RSDLGAVQNRFNSAITNLGNTVNNLTSARSRIEDSDYATEVSNMSRAQILQQAGTSVLAQANQVP
QNVLSLLR (SEQ ID NO: 24)
Salmonella typhimurium flagellin fragment Flic-17a
ANRFTANIKGLTQASRNANDGISIAQTTEGALNEINNNLQRVRELAVQSANSTNSQSDLDSIQAEIT
QRLNEIDRVSGQTQFNGVKVLAQDNTLTIQVGANDGETIDIDLKQINSQTLGLDgaagKIDAALAQV
DTLRSDLGAVQNRFNSAITNLGNTVNNLTSARSRIEDSDYATEVSNMSRAQI (SEQ ID NO: 25)
Salmonella typhimurium flagellin fragment Flic-18a
ANRFTANIKGLTQASRNANDGISIAQTTEGALNEINNNLQRVRELAVQSANSTNSQSDLDSIQAEIT
QRLNEIDRVSGQTQFNGVKVLAQDNTLTIQVGANDGETIDIDLKQINSQTLGLDTLNgaagKIDAAL
AQVDTLRSDLGAVQNRFNSAITNLGNTVNNLTSARSRIEDSDYATEVSNMSRAQI (SEQ ID NO:
26)
Salmonella typhimurium flagellin fragment Flic-19a
ANRFTANIKGLTQASRNANDGISIAQTTEGALNEINNNLQRVRELAVQSANSTNSQSDLDSIQAEIT
QRLNEIDRVSGQTQFNGVKVLAQDNTLTIQVGANDGETIDIDLKQINSQTLGLDgaagATTTENPLQ
KIDAALAQVDTLRSDLGAVQNRFNSAITNLGNTVNNLTSARSRIEDSDYATEVSNMSRAQI (SEQ
ID NO: 27)
Salmonella typhimurium flagellin fragment Flic-20a
ANRFTANIKGLTQASRNANDGISIAQTTEGALNEINNNLQRVRELAVQSANSTNSQSDLDSIQAEIT
QRLNEIDRVSGQTQFNGVKVLAQDNTLTIQVGANDGETIDIDLKQINSQTLGLDgaagKIDAALAQV
DTLRSDLGAVQNRFNSAITNLGNTVNNLTSARSRIEDSDYATEVSNMSRAQILQQAGTSVLAQAN
QVPQNVLSLLR (SEQ ID NO: 28)
Salmonella typhimurium flagellin fragment Flic-21a
ANRFTANIKGLTQASRNANDGISIAQTTEGALNEINNNLQRVRELAVQSANSTNSQSDLDSIQAEIT
QRLNEIDRVSGQTQFNGVKVLAQDNTLTIQVGANDGETIDIDLKQINSQTLGLDTLNgaagATTTEN
PLQKIDAALAQVDTLRSDLGAVQNRFNSAITNLGNTVNNLTSARSRIEDSDYATEVSNMSRAQI
(SEQ ID NO: 29)
Salmonella typhimurium flagellin fragment Flic-22a
ANRFTANIKGLTQASRNANDGISIAQTTEGALNEINNNLQRVRELAVQSANSTNSQSDLDSIQAEIT
QRLNEIDRVSGQTQFNGVKVLAQDNTLTIQVGANDGETIDIDLKQINSQTLGLDTLNgaagKIDAAL
AQVDTLRSDLGAVQNRFNSAITNLGNTVNNLTSARSRIEDSDYATEVSNMSRAQILQQAGTSVLA
QANQVPQNVLSLLR (SEQ ID NO: 30)
Salmonella typhimurium flagellin fragment Flic-23a
ANRFTANIKGLTQASRNANDGISIAQTTEGALNEINNNLQRVRELAVQSANSTNSQSDLDSIQAEIT
QRLNEIDRVSGQTQFNGVKVLAQDNTLTIQVGANDGETIDIDLKQINSQTLGLDgaagATTTENPLQ
KIDAALAQVDTLRSDLGAVQNRFNSAITNLGNTVNNLTSARSRIEDSDYATEVSNMSRAQILQQA
GTSVLAQANQVPQNVLSLLR (SEQ ID NO: 31)
Salmonella typhimurium flagellin fragment Flic-24a
ANRFTANIKGLTQASRNANDGISIAQTTEGALNEINNNLQRVRELAVQSANSTNSQSDLDSIQAEIT
QRLNEIDRVSGQTQFNGVKVLAQDNTLTIQVGANDGETIDIDLKQINSQTLGLDTLNgaagATTTEN
PLQKIDAALAQVDTLRSDLGAVQNRFNSAITNLGNTVNNLTSARSRIEDSDYATEVSNMSRAQIL
QQAGTSVLAQANQVPQNVLSLLR (SEQ ID NO: 32)

In some embodiments, the flagellin polypeptide is secreted from the modified immune cell. In some embodiments, the flagellin polypeptide comprises a signal peptide. The signal peptide (also known as “leader sequence”) is typically inserted at the N-terminus of the protein immediately after the Met initiator. Signal peptides may be cleaved upon export of the flagellin polypeptide from the modified immune cell, forming a mature protein. Signal peptides may be natural or synthetic, and they may be heterologous or homologous to the protein to which they are attached. The choice of signal peptides is wide and is accessible to persons skilled in the art, including, for example, in the online Leader sequence Database maintained by the Department of Biochemistry, National University of Singapore. See Choo et al., BMC Bioinformatics, 6: 249 (2005); and PCT Publication No. WO 2006/081430.

In some embodiments, the flagellin polypeptide is membrane-bound. In some embodiments, the flagellin polypeptide comprises a glycosylphosphatidylinositol (GPI) linker. In some embodiments, the flagellin polypeptide comprises a GPI-anchoring polypeptide sequence at the C-terminus. GPI-anchoring polypeptide sequences are known in the art, including, but not limited to the GPI anchor sequence of human LFA3, CD44, CD59, human Fcγ receptor III (CD16b). See Kueng et al., J Virol, 2007, 81(16):8666-8676.

In some embodiments, the flagellin polypeptide comprises a transmembrane domain that can be directly or indirectly fused to the flagellin protein or fragment thereof. The transmembrane domain may be derived either from a natural or from a synthetic source. As used herein, a “transmembrane domain” refers to any protein structure that is thermodynamically stable in a cell membrane, preferably a eukaryotic cell membrane. Transmembrane domains compatible for use in the flagellin polypeptide described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the flagellin polypeptide described herein is derived from a Type I single-pass membrane protein. In some embodiments, transmembrane domains from multi-pass membrane proteins may also be compatible for use in the flagellin polypeptide described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the transmembrane domain of the flagellin polypeptide comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD154, KIRDS2, OX40, CD2, CD27, LFA-1 (CD11 a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain is derived from a molecule selected from the group consisting of CD8a, CD4, CD28, 4-1BB, CD80, CD86, CD152 and PD1. In some embodiments, the transmembrane domain is derived from CD8a.

Transmembrane domains for use in the flagellin polypeptide described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the flagellin polypeptide comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

The flagellin polypeptide may comprise a hinge region that is located between the flagellin protein or fragment thereof and the transmembrane domain. A hinge region is an amino acid segment that is generally found between two domains of a protein and may allow for flexibility of the protein and movement of one or both of the domains relative to one another. Any amino acid sequence that provides such flexibility and movement of the flagellin protein or fragment thereof relative to the transmembrane domain in the flagellin polypeptide can be used.

The hinge region may contain about 10-100 amino acids, e.g., about any one of 15-75 amino acids, 20-50 amino acids, or 30-60 amino acids. In some embodiments, the hinge region may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge region is a hinge region of a naturally occurring protein. Hinge regions of any protein known in the art to comprise a hinge region are compatible for use in the flagellin polypeptides described herein. In some embodiments, the hinge region is at least a portion of a hinge region of a naturally occurring protein and confers flexibility to the flagellin polypeptide. In some embodiments, the hinge region is derived from CD8a. In some embodiments, the hinge region is a portion of the hinge region of CD8a, e.g., a fragment containing at least 15 (e.g., 20, 25, 30, 35, or 40) consecutive amino acids of the hinge region of CD8a.

Hinge regions of antibodies, such as an IgG, IgA, IgM, IgE, or IgD antibodies, are also compatible for use in the flagellin polypeptide described herein. In some embodiments, the hinge region is the hinge region that joins the constant domains CH1 and CH2 of an antibody. In some embodiments, the hinge region is of an antibody and comprises the hinge region of the antibody and one or more constant regions of the antibody. In some embodiments, the hinge region comprises the hinge region of an antibody and the CH3 constant region of the antibody. In some embodiments, the hinge region comprises the hinge region of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge regions for the flagellin polypeptide. In some embodiments, the hinge region is a peptide linker, such as a (GxS)n linker, wherein x and n, independently can be an integer between 3 and 12, including 3, 4, 5, 6, 7, 8, 9, 10, 11, 12, or more.

In some embodiments, the flagellin polypeptide further comprises an intracellular signaling domain. In some embodiments, the intracellular signaling domain comprises a co-stimulatory signaling domain. The term “co-stimulatory signaling domain,” as used herein, refers to at least a portion of a protein that mediates signal transduction within a cell to induce an immune response such as an effector function. The co-stimulatory signaling domain of the flagellin polypeptide described herein can be a cytoplasmic signaling domain from a co-stimulatory protein, which transduces a signal and modulates responses mediated by immune cells, such as T cells, NK cells, DCs, lymph node (LN) stromal cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains, for example, two copies of the co-stimulatory signaling domain of CD28. In some embodiments, the intracellular signaling domain comprises two or more co-stimulatory signaling domains from different co-stimulatory proteins, such as any two or more co-stimulatory proteins described herein. In some embodiments, the one or more co-stimulatory signaling domains are fused to each other via optional peptide linkers. The one or more co-stimulatory signaling domains may be arranged in any suitable order. Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the flagellin polypeptide described herein. The type(s) of co-stimulatory signaling domain is selected based on factors such as the type of the immune cells in which the flagellin polypeptide would be expressed (e.g., T cells, NK cells, DCs, stromal cells, macrophages, neutrophils, or eosinophils) and the desired immune effector function. Examples of co-stimulatory signaling domains for use in the flagellin polypeptides can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C.

In some embodiments, the one or more co-stimulatory signaling domains are selected from the group consisting of CD27, CD28, 4-1BB (i.e., CD137), OX40, DAP10, CD30, CD40, CD3, lymphocyte function-associated antigen-1(LFA-1), CD2, CD7, LIGHT, NKG2C, B7-H3 and ligands that specially bind to CD83.

In some embodiments, the intracellular signaling domain in the flagellin polypeptide comprises a co-stimulatory signaling domain derived from CD28. In some embodiments, the intracellular signaling domain in the flagellin polypeptide comprises a co-stimulatory signaling domain derived from 4-1BB (i.e., CD137). In some embodiments, the intracellular signaling domain in the flagellin polypeptide comprises a co-stimulatory signaling domain derived from OX40. In some embodiments, the intracellular signaling domain in the flagellin polypeptide comprises a co-stimulatory signaling domain derived from DAP10. In some embodiments, the intracellular signaling domain in the flagellin polypeptide comprises a co-stimulatory signaling domain derived from CD27.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

In some embodiments, wherein the flagellin polypeptide comprises a GPI linker or a transmembrane domain, the flagellin polypeptide further comprises a signal peptide that targets the flagellin polypeptide to the secretory pathway of the cell (e.g., ER) and will allow for integration and anchoring of the flagellin polypeptide into the lipid bilayer of the host cell. Signal peptides including signal sequences of naturally occurring proteins or synthetic, non-naturally occurring signal sequences, which are compatible for use in the transmembrane flagellin polypeptides described herein will be evident to one of skill in the art. In some embodiments, the signal peptide is derived from a molecule selected from the group consisting of CD8a, GM-CSF receptor α, IL-3, and IgG1 heavy chain. In some embodiments, the signal peptide is derived from CD8a.

In some embodiments, a peptide tag (typically a short peptide sequence able to be recognized by available antisera or compounds) may be included for following expression and trafficking of the flagellin polypeptide. A vast variety of tag peptides can be used in the flagellin polypeptide described herein, without limitation, PK tag, FLAG octapeptide, MYC tag, HIS tag (usually a stretch of 4 to 10 histidine residues) and e-tag (U.S. Pat. No. 6,686,152). The tag peptide(s) may be independently positioned at the N-terminus of the protein, at its C-terminus, internally, or at any of these positions when several tags are employed. Tag peptides can be detected by immunodetection assays using anti-tag antibodies.

### Engineered Receptor

Any of the modified immune cells described above may further express an engineered receptor. Exemplary engineered receptor include, but are not limited to, CAR, engineered TCR, and TAC receptors. In some embodiments, the engineered receptor comprises an extracellular domain that specifically binds to an antigen (e.g., a tumor antigen), a transmembrane domain, and an intracellular signaling domain. In some embodiments, the intracellular signaling domain comprises a primary intracellular signaling domain and/or a co-stimulatory domain. In some embodiments, the intracellular signaling domain comprises an intracellular signaling domain of a TCR co-receptor. In some embodiments, the engineered receptor is encoded by the heterologous nucleic acid sequence encoding the flagellin polypeptide. In some embodiments, the engineered receptor is encoded by a second heterologous nucleic acid operably linked to a promoter (such as a constitutive promoter or an inducible promoter). In some embodiments, the engineered receptor is introduced to the modified immune cell by inserting proteins into the cell membrane while passing cells through a microfluidic system, such as CELL SQUEEZE® (see, for example, U.S. Patent Application Publication No. 20140287509). The engineered receptor may enhance the function of the modified immune cell, such as by targeting the modified immune cell, by transducing signals, and/or by enhancing cytotoxicity of the modified immune cell. In some embodiments, the modified immune cell does not express an engineered receptor, such as CAR, TCR, or TAC receptor.

In some embodiments, the engineered receptor comprises one or more specific binding domains that target at least one tumor antigen, and one or more intracellular effector domains, such as one or more primary intracellular signaling domains and/or co-stimulatory domains.

In some embodiments, the engineered receptor is a chimeric antigen receptor (CAR). Many chimeric antigen receptors are known in the art and may be suitable for the modified immune cell of the present invention. CARs can also be constructed with a specificity for any cell surface marker by utilizing antigen binding fragments or antibody variable domains of, for example, antibody molecules. Any method for producing a CAR may be used herein. See, for example, U.S. Pat. Nos. 6,410,319, 7,446,191, 7,514,537, 9,765,342B2, WO 2002/077029, WO2015/142675, US2010/065818, US 2010/025177, US 2007/059298, WO2017025038A1, and Berger C. et al., J. Clinical Investigation 118: 1 294-308 (2008), which are hereby incorporated by reference. In some embodiments, the modified immune cell is a CAR-T cell.

CARs of the present invention comprise an extracellular domain comprising at least one targeting domain that specifically binds at least one tumor antigen, a transmembrane domain, and an intracellular signaling domain. In some embodiments, the intracellular signaling domain generates a signal that promotes an immune effector function of the CAR-containing cell, e.g., a CAR-T cell. “Immune effector function or immune effector response” refers to function or response, e.g., of an immune effector cell, that enhances or promotes an immune attack of a target cell. For example, an immune effector function or response may refer to a property of a T or NK cell that promotes killing or the inhibition of growth or proliferation, of a target cell. Examples of immune effector function, e.g., in a CAR-T cell, include cytolytic activity (such as antibody-dependent cellular toxicity, or ADCC) and helper activity (such as the secretion of cytokines). In some embodiments, the CAR has an intracellular signaling domain with an attenuated immune effector function. In some embodiments, the CAR has an intracellular signaling domain having no more than about any of 90%, 80%, 70%, 60%, 50%, 40%, 30%, 20%, 10% or less of an immune effector function (such as cytolytic function against target cells) compared to a CAR having a full-length and wildtype CD3ζ and optionally one or more co-stimulatory domains. In some embodiments, the intracellular signaling domain generates a signal that promotes proliferation and/or survival of the CAR containing cell. In some embodiments, the CAR comprises one or more intracellular signaling domains selected from the signaling domains of CD28, CD137, CD3, CD27, CD40, ICOS, GITR, and OX40. The signaling domain of a naturally occurring molecule can comprise the entire intracellular (i.e., cytoplasmic) portion, or the entire native intracellular signaling domain, of the molecule, or a fragment or derivative thereof.

In some embodiments, the intracellular signaling domain of a CAR comprises a primary intracellular signaling domain. “Primary intracellular signaling domain” refers to cytoplasmic signaling sequence that acts in a stimulatory manner to induce immune effector functions. In some embodiments, the primary intracellular signaling domain contains a signaling motif known as Immunoreceptor Tyrosine-based Activation Motif, or ITAM. In some embodiments, the primary intracellular signaling domain comprises a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma R IIa, DAP10, and DAP 12. In some embodiments, the primary intracellular signaling domain comprises a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma R IIa, DAP10, and DAP 12. The nonfunctional or attenuated signaling domain can be a mutant signaling domain having a point mutation, insertion or deletion that attenuates or abolishes one or more immune effector functions, such as cytolytic activity or helper activity, including antibody-dependent cellular toxicity (ADCC). In some embodiments, the CAR comprises a nonfunctional or attenuated CD3 zeta (i.e. CD3ζ or CD3z) signaling domain. In some embodiments, the intracellular signaling domain does not comprise a primary intracellular signaling domain. An attenuated primary intracellular signaling domain may induce no more than about any of 90%, 80%, 70%, 60%, 50%, 40%, 30%, 20%, 10% or less of an immune effector function (such as cytolytic function against target cells) compared to CARs having the same construct, but with the wildtype primary intracellular signaling domain.

In some embodiments, the intracellular signaling domain of a CAR comprises one or more (such as any of 1, 2, 3, or more) co-stimulatory domains. “Co-stimulatory domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such co-stimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some embodiments, the CAR comprises a single co-stimulatory domain. In some embodiments, the CAR comprises two or more co-stimulatory domains. In some embodiments, the intracellular signaling domain comprises a functional primary intracellular signaling domain and one or more co-stimulatory domains. In some embodiments, the CAR does not comprise a functional primary intracellular signaling domain (such as CD3ζ). In some embodiments, the CAR comprises an intracellular signaling domain consisting of or consisting essentially of one or more co-stimulatory domains. In some embodiments, the CAR comprises an intracellular signaling domain consisting of or consisting essentially of a nonfunctional or attenuated primary intracellular signaling domain (such as a mutant CD3ζ) and one or more co-stimulatory domains. Upon binding of the targeting domain to tumor antigen, the co-stimulatory domains of the CAR may transduce signals for enhanced proliferation, survival and differentiation of the engineered immune cells having the CAR (such as T cells), and inhibit activation induced cell death. In some embodiments, the one or more co-stimulatory signaling domains are derived from one or more molecules selected from the group consisting of CD27, CD28, 4-1BB (i.e., CD137), OX40, CD30, CD40, CD3, lymphocyte function-associated antigen-1(LFA-1), CD2, CD7, LIGHT, NKG2C, B7-H3 and ligands that specially bind to CD83.

In some embodiments, the intracellular signaling domain of the CAR comprises a co-stimulatory signaling domain derived from CD28. In some embodiments, the intracellular signaling domain comprises a cytoplasmic signaling domain of CD3ζ and a co-stimulatory signaling domain of CD28. In some embodiments, the intracellular signaling domain in the chimeric receptor of the present application comprises a co-stimulatory signaling domain derived from 4-1BB (i.e., CD137). In some embodiments, the intracellular signaling domain comprises a cytoplasmic signaling domain of CD3ζ and a co-stimulatory signaling domain of 4-1BB.

In some embodiments, the intracellular signaling domain of the CAR comprises a co-stimulatory signaling domain of CD28 and a co-stimulatory signaling domain of 4-1BB. In some embodiments, the intracellular signaling domain comprises a cytoplasmic signaling domain of CD3ζ, a co-stimulatory signaling domain of CD28, and a co-stimulatory signaling domain of 4-1BB. In some embodiments, the intracellular signaling domain comprises a polypeptide comprising from the N-terminus to the C-terminus: a co-stimulatory signaling domain of CD28, a co-stimulatory signaling domain of 4-1BB, and a cytoplasmic signaling domain of CD3ζ.

In some embodiments, the targeting domain of the CAR is an antibody or an antibody fragment, such as an scFv, a Fv, a Fab, a (Fab′)₂, a single domain antibody (sdAb), or a V_{H}H domain. In some embodiments, the targeting domain of the CAR is a ligand or an extracellular portion of a receptor that specifically binds to a tumor antigen. In some embodiments, the one or more targeting domains of the CAR specifically bind to a single tumor antigen. In some embodiments, the CAR is a bispecific or multispecific CAR with targeting domains that bind two or more tumor antigens. In some embodiments, the tumor antigen is selected from the group consisting of CD19, BCMA, NY-ESO-1, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance, and combinations thereof.

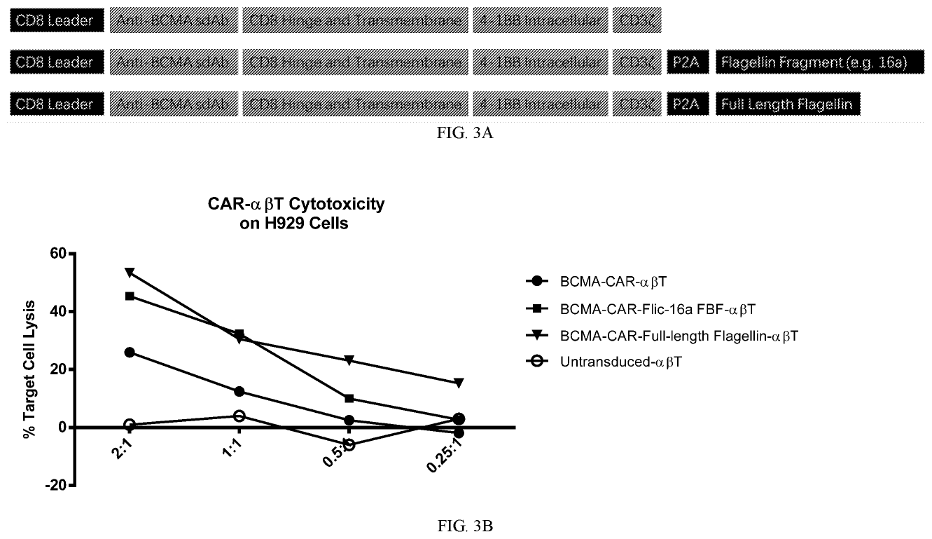

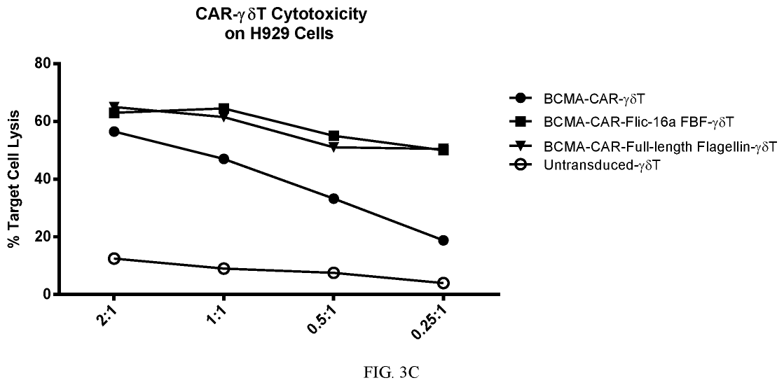

In some embodiments, the CAR is an anti-BCMA CAR. A wide variety of antigen binding domain sequences can be used as the targeting domains of the CAR. See, e.g., WO2017/025038, which is incorporated herein in its entirety. An exemplary CAR construct is shown in FIG. 3A. In some embodiments, the anti-BCMA CAR comprises from the N-terminus to the C-terminus: a CD8 leader, an anti-BCMA sdAb, a CD8 hinge, a CD8 transmembrane, a 4-1BB intracellular co-stimulatory domain, and a CD3ζ intracellular signaling domain. In some embodiments, the anti-BCMA CAR comprises the amino acid sequence of SEQ ID NO: 33.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain of the CAR is a CD4, CD3, CD8a, or CD28 transmembrane domain. In some embodiments, the transmembrane domain of the CAR comprises a transmembrane domain of CD8a.

In some embodiments, the extracellular domain is connected to the transmembrane domain by a hinge region. In one embodiment, the hinge region comprises the hinge region of CD8a.

In some embodiments, the CAR comprises a signal peptide, such as a CD8α.

In some embodiments, the engineered receptor is a modified T-cell receptor. In some embodiments, the engineered TCR is specific for a tumor antigen. In some embodiments, the tumor antigen is selected from the group consisting of CD19, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance. In some embodiments, the tumor antigen is derived from an intracellular protein of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (HER2, NY-BR1, for example). Any of the TCRs known in the art may be used in the present application. In some embodiments, the TCR has an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001). In some embodiments, the modified immune cell is a TCR-T cell.

The TCR receptor complex is an octomeric complex formed by variable TCR receptor α and β chains (γ and δ chains on case of γδ T cells) with three dimeric signaling modules CD3δ/ε, CD3γ/ε and CD247 (T-cell surface glycoprotein CD3 zeta chain) ζ/ζ or ζ/η. Ionizable residues in the transmembrane domain of each subunit form a polar network of interactions that hold the complex together. TCR complex has the function of activating signaling cascades in T cells.

In some embodiments, the engineered receptor is an engineered TCR comprising one or more T-cell receptor (TCR) fusion proteins (TFPs). Exemplary TFPs have been described, for example, in US20170166622A1, which is incorporated herein by reference. In some embodiments, the TFP comprises an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TFP comprises a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TFP comprises a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some embodiments, the TFP comprising a TCR subunit comprising at least a portion of a TCR extracellular domain, and a TCR intracellular domain comprising a stimulatory domain from an intracellular signaling domain of CD3 epsilon; and an antigen binding domain, wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of CD3 gamma; and an antigen binding domain wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of CD3 delta; and an antigen binding domain, wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of TCR alpha; and an antigen binding domain wherein the TCR subunit and the antigen binding domain are operatively linked, and wherein the TFP incorporates into a TCR when expressed in a T cell.

In some embodiments, the TFP comprises a TCR subunit comprising at least a portion of a TCR extracellular domain, and a TCR intracellular domain comprising a stimulatory domain from an intracellular signaling domain of TCR beta; and an antigen binding domain wherein the TCR subunit and the antigen binding domain are operatively linked, and wherein the TFP incorporates into a TCR when expressed in a T cell.

In some embodiments, the engineered receptor is a T-cell antigen coupler (TAC) receptor. Exemplary TAC receptors have been described, for example, in US20160368964A1, which is incorporated herein by reference. In some embodiments, the TAC comprises a targeting domain, a TCR-binding domain that specifically binds a protein associated with the TCR complex, and a T-cell receptor signaling domain. In some embodiments, the targeting domain is an antibody fragment, such as scFv or VHH, which specifically binds to a tumor antigen. In some embodiments, the targeting domain is a designed Ankyrin repeat (DARPin) polypeptide. In some embodiments, the tumor antigen is selected from the group consisting of CD19, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, mesothelin, PSMA, ROR1, WT1, and other tumor antigens with clinical significance. In some embodiments, the protein associated with the TCR complex is CD3, such as CD3E. In some embodiments, the TCR-binding domain is a single chain antibody, such as scFv, or a V_{H}H. In some embodiments, the TCR-binding domain is derived from UCHT1. In some embodiments, the TAC receptor comprises a cytosolic domain and a transmembrane domain. In some embodiments, the T-cell receptor signaling domain comprises a cytosolic domain derived from a TCR co-receptor. Exemplary TCR co-receptors include, but are not limited to, CD4, CD8, CD28, CD45, CD4, CD5, CD9, CD16, CD22, CD33, CD37, CD64, CD80, CD86, CD134, CD137 and CD 154. In some embodiments, the TAC receptor comprises a transmembrane domain and a cytosolic domain derived from CD4. In some embodiments, the TAC receptor comprises a transmembrane domain and a cytosolic domain derived from CD8 (such as CD8a).

T cell co-receptors are expressed as membrane protein on T cells. They can provide stabilization of the TCR: peptide: MEC complex and facilitate signal transduction. The two subtypes of T cell co-receptor, CD4 and CD8, display strong specificity for particular MEC classes. The CD4 co-receptor can only stabilize TCR: MEC II complexes while the CD8 co-receptor can only stabilize the TCR: MEC I complex. The differential expression of CD4 and CD8 on different T cell types results in distinct T cell functional subpopulations. CD8+ T cells are cytotoxic T cells.

CD4 is a glycoprotein expressed on the surface of immune cells such as T helper cells, monocytes, macrophages, and dendritic cells. CD4 has four immunoglobulin domains (D₁ to D₄) exposed on the extracellular cell surface. CD4 contains a special sequence of amino acids on its short cytoplasmic/intracellular tail, which allow CD4 tail to recruit and interact with the tyrosine kinase Lck. When the TCR complex and CD4 each bind to distinct regions of the MEC II molecule, the close proximity between the TCR complex and CD4 allows Lck bound to the cytoplasmic tail of CD4 to tyrosine-phosphorylate the Immunoreceptor Tyrosine Activation Motifs (ITAM) on the cytoplasmic domains of CD3, thus amplifying TCR generated signal.

CD8 is a glycoprotein of either a homodimer composed of two α chains (less common), or a heterodimer composed of one α and one β chain (more common), each comprising an immunoglobulin variable (IgV)-like extracellular domain connected to the membrane by a thin stalk, and an intracellular tail. CD8 is predominantly expressed on the surface of cytotoxic T cells, but can also be found on natural killer cells, cortical thymocytes, and dendritic cells. The CD8 cytoplasmic tail interacts with Lck, which phosphorylates the cytoplasmic CD3 and ζ-chains of the TCR complex once TCR binds its specific antigen. Tyrosine-phosphorylation on the cytoplasmic CD3 and ζ-chains initiates a cascade of phosphorylation, eventually leading to gene transcription.

In some embodiments, the modified immune cell expresses more than one engineered receptors, such as any combination of CAR, TCR, TAC receptor.

In some embodiments, the engineered receptor (such as CAR, TCR, or TAC) expressed by the modified immune cell targets one or more tumor antigens. Tumor antigens are proteins that are produced by tumor cells that can elicit an immune response, particularly T-cell mediated immune responses. The selection of the targeted antigen of the invention will depend on the particular type of cancer to be treated. Exemplary tumor antigens include, for example, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B cell differentiation antigens such as CD 19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

### Nucleic Acids

The modified immune cells described herein comprises one or more heterologous nucleic acids sequence(s) encoding any one of the flagellin polypeptides and/or engineered receptors described herein.

In some embodiments, there is provided an isolated nucleic acid comprising a nucleic acid sequence encoding any one of the flagellin polypeptides described herein. In some embodiments, there is provided an isolated nucleic acid comprising a nucleic acid sequence encoding any one of the engineered receptors described herein. In some embodiments, the nucleic acid is a DNA. In some embodiments, the nucleic acid is a RNA. In some embodiments, the nucleic acid is linear. In some embodiments, the nucleic acid is circular.

The nucleic acid sequence encoding the flagellin polypeptide and/or the nucleic acid encoding the engineered receptor may be operably linked to one or more regulatory sequences. Exemplary regulatory sequences that control the transcription and/or translation of a coding sequence are known in the art and may include, but not limited to, a promoter, additional elements for proper initiation, regulation and/or termination of transcription (e.g. polyA transcription termination sequences), mRNA transport (e.g. nuclear localization signal sequences), processing (e.g. splicing signals), stability (e.g. introns and non-coding 5′ and 3′ sequences), translation (e.g. an initiator Met, tripartite leader sequences, IRES ribosome binding sites, signal peptides, etc.), and insertion site for introducing an insert into the viral vector. In some embodiments, the regulatory sequence is a promoter, a transcriptional enhancer and/or a sequence that allows for proper expression of the flagellin polypeptide and/or the engineered receptor.

The term “regulatory sequence” or “control sequence” refers to a DNA sequence that affects the expression of a coding sequence to which it is operably linked. The nature of such regulatory sequences differs depending upon the host organism. In prokaryotes, regulatory sequences generally include promoters, ribosomal binding sites, and terminators. In eukaryotes, regulatory sequences include promoters, terminators and, in some instances, enhancers, transactivators or transcription factors.

The term “operably linked” refers to a juxtaposition wherein the components so described are in a relationship permitting them to function in their intended manner. A regulatory sequence “operably linked” to a coding sequence is ligated in such a way that expression of the coding sequence is achieved under conditions compatible with the regulatory sequences.

As used herein, a “promoter” or a “promoter region” refers to a segment of DNA or RNA that controls transcription of the DNA or RNA to which it is operatively linked. The promoter region includes specific sequences that are involved in RNA polymerase recognition, binding and transcription initiation. In addition, the promoter includes sequences that modulate recognition, binding and transcription initiation activity of RNA polymerase (i.e., binding of one or more transcription factors). These sequences can be cis acting or can be responsive to trans acting factors. Promoters, depending upon the nature of the regulation, can be constitutive or regulated. Regulated promoters can be inducible or environmentally responsive (e.g. respond to cues such as pH, anaerobic conditions, osmoticum, temperature, light, or cell density). Many such promoter sequences are known in the art. See, for example, U.S. Pat. Nos. 4,980,285; 5,631,150; 5,707,928; 5,759,828; 5,888,783; 5,919,670, and, Sambrook, et al., Molecular Cloning: A Laboratory Manual, 2nd Ed., Cold Spring Harbor Press (1989).

In some embodiments, the nucleic acid sequence encoding the flagellin polypeptide is operably linked to a first promoter. In some embodiments, the nucleic acid sequence encoding the engineered receptor is operably linked to a second promoter. In some embodiments, the nucleic acid sequence encoding the flagellin polypeptide and the nucleic acid sequence encoding the engineered receptor are operably linked to the same promoter. In some embodiments, the nucleic acid sequence encoding the flagellin polypeptide and the nucleic acid sequence encoding the engineered receptor are operably linked to separate promoters.

In some embodiments, the promoter is an endogenous promoter. For example, a nucleic acid encoding the flagellin polypeptide and/or the engineered receptor may be knocked-in to the genome of the modified immune cell downstream of an endogenous promoter using any methods known in the art, such as CRISPR/Cas9 method. In some embodiments, the endogenous promoter is a promoter for an abundant protein, such as beta-actin. In some embodiments, the endogenous promoter is an inducible promoter, for example, inducible by an endogenous activation signal of the modified immune cell. In some embodiments, wherein the modified immune cell is a T cell, the promoter is a T cell activation-dependent promoter (such as an IL-2 promoter, an NFAT promoter, or an NFκB promoter). In some embodiments, the promoter is a heterologous promoter.

Varieties of promoters have been explored for gene expression in mammalian cells, and any of the promoters known in the art may be used in the present invention. Promoters may be roughly categorized as constitutive promoters or regulated promoters, such as inducible promoters. In some embodiments, the heterologous nucleic acid sequence encoding the flagellin polypeptide and/or the engineered receptor is operably linked to a constitutive promoter. In some embodiments, the heterologous nucleic acid sequence encoding the flagellin polypeptide and/or the engineered receptor is operably linked to an inducible promoter. In some embodiments, a constitutive promoter is operably linked to the nucleic acid sequence encoding the flagellin polypeptide, and an inducible promoter is operably linked to the nucleic acid sequence encoding the engineered receptor. In some embodiments, a constitutive promoter is operably linked to the nucleic acid sequence encoding the engineered receptor, and an inducible promoter is operably linked to the nucleic acid sequence encoding the flagellin polypeptide. In some embodiments, a first inducible promoter is operably linked to the nucleic acid sequence encoding the flagellin polypeptide, and a second inducible promoter is operably linked to the nucleic acid sequence encoding the engineered receptor. In some embodiments, the first inducible promoter is inducible by a first inducing condition, and the second inducible promoter is inducible by a second inducing condition. In some embodiments, the first inducing condition is the same as the second inducing condition. In some embodiments, the first inducible promoter and the second inducible promoter are induced simultaneously. In some embodiments, the first inducible promoter and the second inducible promoter are induced sequentially, for example, the first inducible promoter is induced prior to the second inducible promoter, or the first inducible promoter is induced after the second inducible promoter.

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. In some embodiments, the promoter is a hEF1α promoter.

In some embodiments, the promoter is an inducible promoter. Inducible promoters belong to the category of regulated promoters. The inducible promoter can be induced by one or more conditions, such as a physical condition, microenvironment of the modified immune cell, or the physiological state of the modified immune cell, an inducer (i.e., an inducing agent), or a combination thereof. In some embodiments, the inducing condition does not induce the expression of endogenous genes in the modified immune cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the modified immune cell.

In some embodiments, the promoter is inducible by an inducer. In some embodiments, the inducer is a small molecule, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most established system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the therapeutic protein in the present application.

In some embodiments, the inducer is a polypeptide, such as a growth factor, a hormone, or a ligand to a cell surface receptor, for example, a polypeptide that specifically binds a tumor antigen. In some embodiments, the polypeptide is expressed by the modified immune cell. In some embodiments, the polypeptide is encoded by a nucleic acid in the heterologous nucleic acid. Many polypeptide inducers are also known in the art, and they may be suitable for use in the present invention. For example, ecdysone receptor-based gene switches, progesterone receptor-based gene switches, and estrogen receptor based gene switches belong to gene switches employing steroid receptor derived transactivators (WO9637609 and WO9738117 etc.).

In some embodiments, the inducer comprises both a small molecule component and one or more polypeptides. For example, inducible promoters that dependent on dimerization of polypeptides are known in the art, and may be suitable for use in the present invention. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB* and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4): 549-57).

In some embodiments, the promoter is a light-inducible promoter, and the inducing condition is light. Light inducible promoters for regulating gene expression in mammalian cells are also well-known in the art (see, for example, Science 332, 1565-1568 (2011); Nat. Methods 9, 266-269 (2012); Nature 500: 472-476 (2013); Nature Neuroscience 18:1202-1212 (2015)). Such gene regulation systems can be roughly divided into two categories based on their regulations of (1) DNA binding or (2) recruitment of a transcriptional activation domain to a DNA bound protein. For instance, synthetic mammalian blue light controlled transcription system based on melanopsin which, in response to blue light (480 nm), triggers an intracellular calcium increase that result in calcineurin-mediated mobilization of NFAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem. Biol. 10(3):196-202 (2014)). Additionally, the red light induced interaction of photoreceptor phytochrome B (PhyB) and phytochrome-interacting factor 6 (PIF6) of Arabidopsis thaliana was exploited for a red light triggered gene expression regulation. Furthermore, ultraviolet B (UVB)-inducible gene expression system were also developed and proven to be efficient in target gene transcription in mammalian cells (Chapter 25 of Gene and Cell Therapy: Therapeutic Mechanisms and Strategies, Fourth Edition CRC Press, Jan. 20, 2015). Any of the light-inducible promoters described herein may be used to drive expression of the therapeutic protein in the present invention.

In some embodiments, the promoter is a light-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IPTG for use with the lac operon, caged toyocamycin for ribozyme-mediated gene expression, caged doxycycline for use with the Tet-on system, and caged Rapalog for light mediated FKBP/FRB dimerization have been developed (see, for example, Curr Opin Chem Biol. 16(3-4): 292-299 (2012)).

In some embodiments, the promoter is a radiation-inducible promoter, and the inducing condition is radiation, such as ionizing radiation. Radiation inducible promoters are also known in the art to control transgene expression. Alteration of gene expression occurs upon irradiation of cells. For example, a group of genes known as “immediate early genes” can react promptly upon ionizing radiation. Exemplary immediate early genes include, but are not limited to, Erg-1, p21/WAF-1, GADD45alpha, t-PA, c-Fos, c-Jun, NF-kappaB, and AP1. The immediate early genes comprise radiation responsive sequences in their promoter regions. Consensus sequences CC(A/T)₆GG have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6(7):1005-12 (2007) and Chapter 25 of Gene and Cell Therapy: Therapeutic Mechanisms and Strategies, Fourth Edition CRC Press, Jan. 20, 2015.

In some embodiments, the promoter is a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hsp10 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appears to be one of the most highly-efficient heat inducible promoters. Huang et al reported that after introduction of hsp70B-EGFP, hsp70B-TNFalpha and hsp70B-IL12 coding sequences, tumor cells expressed extremely high transgene expression upon heat treatment, while in the absence of heat treatment, the expression of transgenes were not detected. And tumor growth was delayed significantly in the IL12 transgene plus heat treated group of mice in vivo (Cancer Res. 60:3435 (2000)). Another group of scientists linked the HSV-tk suicide gene to hsp70B promoter and test the system in nude mice bearing mouse breast cancer. Mice whose tumor had been administered the hsp70B-HSVtk coding sequence and heat treated showed tumor regression and a significant survival rate as compared to no heat treatment controls (Hum. Gene Ther. 11:2453 (2000)). Additional heat inducible promoters known in the art can be found in, for example, Chapter 25 of Gene and Cell Therapy: Therapeutic Mechanisms and Strategies, Fourth Edition CRC Press, Jan. 20, 2015. Any of the heat-inducible promoters discussed herein may be used to drive the expression of the therapeutic protein of the present invention.

In some embodiments, the promoter is inducible by a redox state. Exemplary promoters that are inducible by redox state include inducible promoter and hypoxia inducible promoters. For instance, Post D E et al developed hypoxia-inducible factor (HIF) responsive promoter which specifically and strongly induce transgene expression in RIF-active tumor cells (Gene Ther. 8: 1801-1807 (2001); Cancer Res. 67: 6872-6881 (2007)).

In some embodiments, the promoter is inducible by the physiological state, such as an endogenous activation signal, of the modified immune cell. In some embodiments, wherein the modified immune cell is a T cell, the promoter is a T cell activation-dependent promoter, which is inducible by the endogenous activation signal of the modified T cell. In some embodiments, the modified T cell is activated by an inducer, such as phorbol myristate acetate (PMA), ionomycin, or phytohaemagglutinin. In some embodiments, the modified T cell is activated by recognition of a tumor antigen on the tumor cells via the engineered receptor (such as CAR, TCR or TAC). In some embodiments, the T cell activation-dependent promoter is an IL-2 promoter. In some embodiments, the T cell activation-dependent promoter is an NFAT promoter. In some embodiments, the T cell activation-dependent promoter is a NFκB promoter.

The heterologous nucleic acid sequences(s) described herein can be present in a heterologous gene expression cassette, which comprises one or more protein-coding sequences and optionally one or more promoters. In some embodiments, the heterologous gene expression cassette comprises a single protein-coding sequence. In some embodiments, the heterologous gene expression cassette comprises two or more protein-coding sequences driven by a single promoter (i.e., polycistronic). In some embodiments, the heterologous gene expression cassette further comprises one or more regulatory sequences (such as 5′UTR, 3′UTR, enhancer sequence, IRES, transcription termination sequence), recombination sites, one or more selection markers (such as antibiotic resistance gene, reporter gene, etc.), signal sequence, or combinations thereof.

In some embodiments, there is provided a vector comprising any one of the nucleic acids encoding the flagellin polypeptides and/or the engineered receptors described herein. In some embodiments, there is provided a vector comprising a first nucleic acid sequence encoding any one of the flagellin polypeptides described herein and a second nucleic acid sequence encoding any one of the engineered receptors described herein. In some embodiments, the first nucleic acid sequence encoding the flagellin polypeptide is fused to the second nucleic acid sequence encoding the engineered receptor via a third nucleic acid sequence encoding a self-cleavable linker, such as P2A, T2A, E2A, or F2A peptide. In some embodiments, there is provided a composition comprising a first vector comprising a first nucleic acid sequence encoding any one of the flagellin polypeptides described herein, and a second vector comprising a second nucleic acid sequence encoding any one of the engineered receptors described herein.

In some embodiments, there is provided a vector comprising a first nucleic acid sequence encoding a CAR (e.g., an anti-BCMA CAR) and a second nucleic acid sequence encoding a full-length flagellin protein, wherein the first nucleic acid sequence is fused to the second nucleic acid sequence via a third nucleic acid sequence encoding a self-cleavable linker, such as P2A. In some embodiments, there is provided a vector comprising a first nucleic acid sequence encoding a CAR (e.g., an anti-BCMA CAR) and a second nucleic acid sequence encoding a flagellin polypeptide comprising the amino acid sequence of SEQ ID NO: 24, wherein the first nucleic acid sequence is fused to the second nucleic acid sequence via a third nucleic acid sequence encoding a self-cleavable linker, such as P2A. In some embodiments, there is provided a vector comprising a first nucleic acid sequence encoding a CAR (e.g., an anti-BCMA CAR) and a second nucleic acid sequence encoding a flagellin polypeptide comprising the amino acid sequence of SEQ ID NO: 32, wherein the first nucleic acid sequence is fused to the second nucleic acid sequence via a third nucleic acid sequence encoding a self-cleavable linker, such as P2A.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In general, a suitable vector contains an origin of replication functional in at least one organism, a promoter sequence, convenient restriction endonuclease sites, and one or more selectable markers. The term “vector” should also be construed to include non-plasmid and non-viral compounds which facilitate transfer of nucleic acid into cells, such as, for example, polylysine compounds, liposomes, and the like.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the modified immune cell in vitro or ex vivo. A number of retroviral systems are known in the art. In some embodiments, adenovirus vectors are used. In some embodiments, lentivirus vectors are used. In some embodiments, self-inactivating lentiviral vectors are used. For example, self-inactivating lentiviral vectors can be packaged with protocols known in the art. The resulting lentiviral vectors can be used to transduce a mammalian cell (such as human T cells) using methods known in the art.

In some embodiments, the vector is a non-viral vector, such as a plasmid, or an episomal expression vector.

In some embodiments, the vector is an expression vector. “Expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in eukaryotic cells are well known to those skilled in the art. In the case of eukaryotic cells they comprise normally promoters ensuring initiation of transcription and optionally poly-A signals ensuring termination of transcription and stabilization of the transcript. Examples of regulatory elements permitting expression in eukaryotic host cells are AOX1 or GAL1 promoter in yeast or the CMV-, SV40-, RSV-promoter (Rous sarcoma virus), CMV-enhancer, SV40-enhancer or a globin intron in mammalian and other animal cells. Furthermore, depending on the expression system used leader sequences capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the nucleic acid sequence can encode a fusion protein including an N-terminal identification peptide imparting desired characteristics, e.g., stabilization or simplified purification of expressed recombinant product. Suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al., Cancer Immunol Immunother (2001) 50(3), 141-150) or pSPORT1 (GIBCO BRL).

### Methods of Preparation

The present application also provides methods of preparing any one of the modified immune cells described herein.

In some embodiments, there is provided a method of producing a modified immune cell, comprising: introducing into a precursor immune cell a first nucleic acid sequence encoding any one of the flagellin polypeptides described herein. In some embodiments, the precursor immune cell is selected from the group consisting of a cytotoxic T cell, a helper T cell, a natural killer (NK) cell, an NK-T cell, an iNK-T cell, an NK-T like cell, an αβT cell and a γδT cell. In some embodiments, the precursor immune cell is a cytotoxic T cell. In some embodiments, the precursor immune cell is a γδT cell. In some embodiments, the precursor immune cell is a tumor-infiltrating T cell or DC-activated T cell. In some embodiments, the precursor immune cell comprises any one of the engineered receptors described herein. In some embodiments, the method further comprises introducing into the precursor immune cell a second nucleic acid encoding any one of the engineered receptors described herein. In some embodiments, the engineered receptor is a chimeric antigen receptor (CAR). In some embodiments, the engineered receptor is a modified T-cell receptor (TCR). In some embodiments, the engineered receptor is a T-cell antigen coupler (TAC) receptor. In some embodiments, the first nucleic acid sequence and the second nucleic acid sequence are operably linked to the same promoter. In some embodiments, the first nucleic acid sequence and the second nucleic acid sequence are operably linked to separate promoters. In some embodiments, the first nucleic acid and the second nucleic acid are on the same vector. In some embodiments, the first nucleic acid and the second nucleic acid are on separate vectors. In some embodiments, the vector is a viral vector. In some embodiments, the viral vector is selected from the group consisting of an adenoviral vector, an adeno-associated virus vector, a retroviral vector, a lentiviral vector, a herpes simplex viral vector, and derivatives thereof. In some embodiments, the vector is a non-viral vector. In some embodiments, the vector is an episomal expression vector. In some embodiments, the method further comprises isolating or enriching immune cells comprising the first nucleic acid sequence and/or the second nucleic acid sequence. In some embodiments, the method further comprises formulating the modified immune cells with at least one pharmaceutically acceptable carrier.

In some embodiments, there is provided an isolated host cell comprising any one of the nucleic acids or vectors described herein. The host cells may be useful in expression or cloning of the flagellin polypeptides and/or the engineered receptors, nucleic acids or vectors encoding the flagellin polypeptides and/or the engineered receptors. Suitable host cells can include, without limitation, prokaryotic cells, fungal cells, yeast cells, or higher eukaryotic cells such as mammalian cells. In some embodiments, the host cells comprise a first vector encoding a first polypeptide and a second vector encoding a second polypeptide. In some embodiments, the host cells comprise a single vector comprising isolated nucleic acids encoding a first polypeptide and a second polypeptide.

The precursor immune cells can be prepared using a variety of methods known in the art. For example, primary immune cells, such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, immune cells (such as T cells) can be obtained from a unit of blood collected from an individual using any number of techniques known in the art, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS), or a wash solution lacking divalent cations, such as calcium and magnesium. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, primary T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA, and CD45RO cells, can be further isolated by positive or negative selection techniques. For example, in one embodiment, T cells are isolated by incubation with anti-CD3/anti-CD28 (i.e., 3×28)-conjugated beads, such as DYNABEADS®M-450 CD3/CD28 T, for a time period sufficient for positive selection of the desired T cells.

In some embodiments, a T cell population may further be enriched by negative selection using a combination of antibodies directed to surface markers unique to the negatively selected cells. For example, one method involves cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar methods of selection.

Methods of introducing vectors or nucleic acids into a host cell (such as a precursor immune cell) are known in the art. The vectors or nucleic acids can be transferred into a host cell by physical, chemical, or biological methods.

Physical methods for introducing the vector(s) or nucleic acid(s) into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector is introduced into the cell by electroporation.

Biological methods for introducing the vector(s) or nucleic acid(s) into a host cell include the use of DNA and RNA vectors. Viral vectors have become the most widely used method for inserting genes into mammalian, e.g., human cells.

Chemical means for introducing the vector(s) or nucleic acid(s) into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, the transduced or transfected precursor immune cell is propagated ex vivo after introduction of the heterologous nucleic acid(s). In some embodiments, the transduced or transfected precursor immune cell is cultured to propagate for at least about any of 1 day, 2 days, 3 days, 4 days, 5 days, 6 days, 7 days, 10 days, 12 days, or 14 days. In some embodiments, the transduced or transfected precursor immune cell is cultured for no more than about any of 1 day, 2 days, 3 days, 4 days, 5 days, 6 days, 7 days, 10 days, 12 days, or 14 days. In some embodiments, the transduced or transfected precursor immune cell is further evaluated or screened to select the modified immune cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)).

Other methods to confirm the presence of the heterologous nucleic acid(s) in the precursor immune cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

### III. Methods of Treatment

One aspect of the present application relates to methods of treating a cancer in an individual, comprising administering to the individual an effective amount of any one of the modified immune cells described herein. The present application contemplates modified immune cells that can be administered either alone or in any combination with another therapy, and in at least some aspects, together with a pharmaceutically acceptable carrier or excipient. In some embodiments, prior to administration, the modified immune cells may be combined with suitable pharmaceutical carriers and excipients that are well known in the art.

In some embodiments, there is provided a method of treating cancer (e.g., solid cancer) in an individual (e.g., human), comprising administering to the individual an effective amount of a pharmaceutical composition comprising a modified immune cell and a pharmaceutically acceptable carrier, wherein the modified immune cell comprises a first heterologous nucleic acid sequence encoding a flagellin polypeptide comprising flagellin or a fragment thereof, wherein the flagellin polypeptide upon expression is capable of binding to a toll-like receptor. In some embodiments, the toll-like receptor is selected from the group consisting of TLR4, TLR5, TLR11, TLR2, TLR3 and TLR9. In some embodiments, the flagellin polypeptide is secreted. In some embodiments, the flagellin polypeptide is membrane bound. In some embodiments, the modified immune cell further comprises an engineered receptor, such as a chimeric antigen receptor (CAR), an engineered TCR, or a T-cell antigen coupler (TAC) receptor. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell. In some embodiments, the modified immune cell further comprises a second heterologous nucleic acid sequence encoding an engineered receptor, such as a CAR, an engineered TCR, or a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters.

In some embodiments, there is provided a method of treating cancer (e.g., solid cancer) in an individual (e.g., human), comprising administering to the individual an effective amount of a pharmaceutical composition comprising a modified immune cell and a pharmaceutically acceptable carrier, wherein the modified immune cell comprises a first heterologous nucleic acid sequence encoding a secreted flagellin polypeptide comprising a flagellin protein or a fragment thereof, wherein the flagellin polypeptide is capable of binding to a toll-like receptor (e.g., TLR5). In some embodiments, the flagellin polypeptide consists of or consists essentially of a flagellin protein or a fragment thereof. In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the modified immune cell is selected from the group consisting of a cytotoxic T cell, a helper T cell, a natural killer (NK) cell, an NK-T cell, an iNK-T cell, an NK-T like cell, an αβT cell, a γδT cell, a tumor-infiltrating T cell and a DC-activated T cell. In some embodiments, the modified immune cell further comprises a second heterologous nucleic acid sequence encoding an engineered receptor, such as a CAR, an engineered TCR, or a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters.

In some embodiments, there is provided a method of treating cancer (e.g., solid cancer) in an individual (e.g., human), comprising administering to the individual an effective amount of a pharmaceutical composition comprising a modified immune cell and a pharmaceutically acceptable carrier, wherein the modified immune cell comprises a first heterologous nucleic acid sequence encoding a flagellin polypeptide comprising a flagellin protein or a fragment thereof and a GPI-anchoring peptide sequence, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the GPI-anchoring peptide sequence is attached to a GPI linker. In some embodiments, the GPI-anchoring peptide sequence is located at the C-terminus of the flagellin polypeptide. In some embodiments, the modified immune cell is selected from the group consisting of a cytotoxic T cell, a helper T cell, a natural killer (NK) T cell, an iNK-T cell, an NK-T like cell, an αβT cell, a γδT cell, a tumor-infiltrating T cell and a DC-activated T cell. In some embodiments, the modified immune cell further comprises a second heterologous nucleic acid sequence encoding an engineered receptor, such as a CAR, an engineered TCR, or a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters.

In some embodiments, there is provided a method of treating cancer (e.g., solid cancer) in an individual (e.g., human), comprising administering to the individual an effective amount of a pharmaceutical composition comprising a modified immune cell and a pharmaceutically acceptable carrier, wherein the modified immune cell comprises a first heterologous nucleic acid sequence encoding a flagellin polypeptide comprising a flagellin protein or a fragment thereof and a transmembrane domain, wherein the flagellin polypeptide is capable of binding to a toll-like receptor (e.g., TLR5). In some embodiments, the flagellin polypeptide comprises Motif N and/or Motif C of a flagellin protein. In some embodiments, the flagellin polypeptide comprises the N-terminal domain and/or the C-terminal domain of a flagellin protein. In some embodiments, the flagellin polypeptide comprises amino acids 1-172 and/or amino acids 418-505 of a flagellin protein, wherein the amino acid sequence numbering is based on SEQ ID NO: 2. In some embodiments, the flagellin polypeptide comprises amino acids 1-172 and/or amino acids 407-494 of SEQ ID NO: 1. In some embodiments, the flagellin polypeptide comprises amino acids 1-172 and/or amino acids 465-553 of SEQ ID NO: 3. In some embodiments, the flagellin polypeptide is a full-length flagellin. In some embodiments, the flagellin polypeptide comprises all or a portion of an amino acid sequence having at least about 85% (e.g., at least about any one of 86%, 87%, 88%, 89%, 90%, 91%, 92%, 93%, 94%, 95%, 96%, 97%, 98%, 99%, or higher) sequence identity to SEQ ID NO: 1, SEQ ID NO: 2 or SEQ ID NO: 3. In some embodiments, the flagellin polypeptide comprises all or a portion of the amino acid sequence of SEQ ID NO: 1, SEQ ID NO: 2, or SEQ ID NO: 3. In some embodiments, the flagellin polypeptide comprises the amino acid sequence of SEQ ID NO: 1. In some embodiments, the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker. In some embodiments, the flagellin polypeptide comprises an amino acid sequence having at least about 85% sequence identity to an amino acid sequence selected from the group consisting of SEQ ID NOs: 12, 14-16, 20, 22-24, and 28-32. In some embodiments, the flagellin polypeptide comprises an amino acid sequence selected from the group consisting of SEQ ID NOs: 12, 14-16, 20, 22-24, and 28-32. In some embodiments, the transmembrane domain is derived from a molecule selected from the group consisting of CD8, CD4, CD28, 4-1BB, CD80, CD86, CD152 and PD1. In some embodiments, the flagellin polypeptide further comprises a hinge domain, such as a hinge domain derived from CD8. In some embodiments, the modified immune cell is selected from the group consisting of a cytotoxic T cell, a helper T cell, a natural killer (NK) T cell, an iNK-T cell, an NK-T like cell, an αβT cell, a γδT cell, a tumor-infiltrating T cell and a DC-activated T cell. In some embodiments, the flagellin polypeptide further comprises an intracellular signaling domain. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, DAP10, CD30, CD40, CD3, LFA-1, CD2, CD7, LIGHT, NKG2C, B7-H3, Ligands of CD83 and combinations thereof. In some embodiments, the pharmaceutical composition is administered to the individual systemically or locally. In some embodiments, the modified immune cell further comprises a second heterologous nucleic acid sequence encoding an engineered receptor, such as a CAR, an engineered TCR, or a TAC receptor. In some embodiments, the first nucleic acid sequence and the second nucleic acid sequence are on the same vector or separate vectors. In some embodiments, the first nucleic acid sequence and the second nucleic acid sequence are operably linked to the same promoter or separate promoters.

In some embodiments, there is provided a method of treating a cancer (e.g., myeloma or plasmacytoma) in an individual (e.g., human), comprising administering to the individual an effective amount of a pharmaceutical composition comprising a modified immune cell and a pharmaceutically acceptable carrier, wherein the modified immune cell comprises a first heterologous nucleic acid sequence encoding a secreted flagellin polypeptide and a second heterologous nucleic acid sequence encoding a chimeric antigen receptor targeting BCMA, wherein the flagellin polypeptide comprises an amino acid sequence having at least about 85% sequence identity to an amino acid sequence selected from the group consisting of SEQ ID NOs: 1, 12, 14-16, 20, 22-24, and 28-32. In some embodiments, the flagellin polypeptide comprises an amino acid sequence selected from the group consisting of SEQ ID NOs: 1, 12, 14-16, 20, 22-24, and 28-32. In some embodiments, the flagellin polypeptide comprises the amino acid sequence of SEQ ID NO: 1. In some embodiments, the flagellin polypeptide comprises the amino acid sequence of SEQ ID NO: 24. In some embodiments, the flagellin polypeptide comprises the amino acid sequence of SEQ ID NO: 32. In some embodiments, the CAR targeting BCMA comprises the amino acid sequence of SEQ ID NO: 33. In some embodiments, the modified immune cell is selected from the group consisting of a cytotoxic T cell, a helper T cell, a natural killer (NK) T cell, an iNK-T cell, an NK-T like cell, an αβT cell, a γδT cell, a tumor-infiltrating T cell and a DC-activated T cell. In some embodiments, the first nucleic acid sequence and the second nucleic acid sequence are on the same vector, e.g., a lentiviral vector. In some embodiments, the first nucleic acid sequence and the second nucleic acid sequence are operably linked to the same promoter or separate promoters.

In some embodiments, the method of treating cancer has one or more of the following biological activities: (1) killing cancer cells; (2) inhibiting proliferation of cancer cells; (3) inducing redistribution of peripheral T cells; (4) inducing immune response in a tumor; (5) reducing tumor size; (6) alleviating one or more symptoms in an individual having cancer; (7) inhibiting tumor metastasis; (8) prolonging survival; (9) prolonging time to cancer progression; (10) preventing, inhibiting, or reducing the likelihood of the recurrence of a cancer; (11) improving quality of life of the individual; (12) facilitating T cell infiltration in tumors, and (13) reducing incidence or burden of preexisting tumor metastasis (such as metastasis to the lymph node). In some embodiments, the method achieves a tumor cell death rate of at least about any of 40%, 50%, 60%, 70%, 80%, 90%, 95%, or more. In some embodiments, the method reduces at least about 10% (including for example at least about any of 20%, 30%, 40%, 60%, 70%, 80%, 90%, or 100%) of the tumor size. In some embodiments, the method inhibits at least about 10% (including for example at least about any of 20%, 30%, 40%, 60%, 70%, 80%, 90%, or 100%) of the metastasis. In some embodiments, the method prolongs the survival of the individual by at least any of 1, 2, 3, 4, 5, 6, 7, 8, 9, 10, 11, 12, 18, 24, or more months. In some embodiments, the method prolongs the time to cancer progression by at least any of 1, 2, 3, 4, 5, 6, 7, 8, 9, 10, 11, 12, 18, 24, or more months.

The methods described herein are suitable for treating a variety of cancers, including both solid cancer and liquid cancer. The methods are applicable to cancers of all stages, including early stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as chemotherapy, surgery, hormone therapy, radiation, gene therapy, immunotherapy (such as T cell therapy), bone marrow transplantation, stem cell transplantation, targeted therapy, cryotherapy, ultrasound therapy, photodynamic therapy, radio-frequency ablation or the like, in an adjuvant setting or a neoadjuvant setting (i.e., the method may be carried out before the primary/definitive therapy). In some embodiments, the method is used to treat an individual who has previously been treated. In some embodiments, the cancer has been refractory to prior therapy. In some embodiments, the method is used to treat an individual who has not previously been treated.

The effective amount of the modified immune cells administered in the methods described herein will depend upon a number of factors, such as the particular type and stage of cancer being treated, the route of administrations, the activity of the flagellin polypeptide and/or the engineered receptors, and the like. Appropriate dosage regimen can be determined by a physician based on clinical factors, including the patient's size, body surface area, age, the particular compound to be administered, sex, time and route of administration, general health, and other drugs being administered concurrently. In some embodiments, that effective amount of the pharmaceutical composition is below the level that induces a toxicological effect (i.e., an effect above a clinically acceptable level of toxicity) or is at a level where a potential side effect can be controlled or tolerated when the pharmaceutical composition is administered to the individual. In some embodiments, the effective amount of the pharmaceutical composition comprises about 10⁵ to about 10¹⁰ modified immune cells.

In some embodiments, the pharmaceutical composition is administered for a single time (e.g. bolus injection). In some embodiments, the pharmaceutical composition is administered for multiple times (such as any of 2, 3, 4, 5, 6, or more times). If multiple administrations, they may be performed by the same or different routes and may take place at the same site or at alternative sites. The pharmaceutical composition may be administered at a suitable frequency, such as from daily to once per year. The optimal dosage and treatment regime for a particular patient can readily be determined by one skilled in the art of medicine by monitoring the patient for signs of disease and adjusting the treatment accordingly.

In some embodiments, the individual to be treated is a mammal. Examples of mammals include, but are not limited to, humans, monkeys, rats, mice, hamsters, guinea pigs, dogs, cats, rabbits, pigs, sheep, goats, horses, cattle and the like. In some embodiments, the individual is a human.

### Pharmaceutical Compositions

Further provided by the present application are pharmaceutical compositions comprising any one of the modified immune cells described herein, and optionally a pharmaceutically acceptable carrier.

The pharmaceutical composition of the present applicant may comprise any number of the modified immune cells. In some embodiments, the pharmaceutical composition comprises a single copy of the modified immune cell. In some embodiments, the pharmaceutical composition comprises at least about any of 1, 10, 100, 1000, 10⁴, 10⁵, 10⁶, 10⁷, 10⁸ or more copies of the modified immune cells. In some embodiments, the pharmaceutical composition comprises a single type of modified immune cell. In some embodiments, the pharmaceutical composition comprises at least two types of modified immune cells, wherein the different types of modified immune cells differ by their cell sources, cell types, expressed chimeric receptors, and/or promoters, etc.

“Carriers” as used herein include pharmaceutically acceptable carriers, excipients, or stabilizers which are nontoxic to the cells or individual being exposed thereto at the dosages and concentrations employed. Often the physiologically acceptable carrier is an aqueous pH buffered solution. Examples of suitable pharmaceutical carriers are well known in the art and include phosphate buffered saline solutions, water, emulsions, such as oil/water emulsions, various types of wetting agents, sterile solutions, etc. Acceptable carriers, excipients, or stabilizers are nontoxic to recipients at the dosages and concentrations employed.

Pharmaceutical compositions comprising such carriers can be formulated by well-known conventional methods. The solvent or diluent is preferably isotonic, hypotonic or weakly hypertonic and has a relatively low ionic strength. Representative examples include sterile water, physiological saline (e.g. sodium chloride), Ringer's solution, glucose, trehalose or saccharose solutions, Hank's solution, and other aqueous physiologically balanced salt solutions (see, for example, the most current edition of Remington: The Science and Practice of Pharmacy, A. Gennaro, Lippincott, Williams&Wilkins).

The pharmaceutical compositions described herein may be administered via any suitable routes. In some embodiments, the pharmaceutical composition is administered parenterally, transdermally (into the dermis), intraluminally, intra-arterially (into an artery), intramuscularly (into muscle), intrathecally or intravenously. In some embodiments, the pharmaceutical composition is administered subcutaneously (under the skin). In some embodiments, the pharmaceutical composition is administered intravenously. In some embodiments, the pharmaceutical composition is administered to the individual via infusion or injection. In some embodiments, the pharmaceutical composition is administered directly to the target site, e.g., by biolistic delivery to an internal or external target site or by catheter to a site in an artery. In some embodiments, the pharmaceutical composition is administered locally, e.g., intratumorally. Administrations may use conventional syringes and needles or any compound or device available in the art capable of facilitating or improving delivery of the active agent(s) in the subject.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. Various virus formulation are available in the art either in frozen, liquid form or lyophilized form (e.g. WO98/02522, WO01/66137, WO03/053463, WO2007/056847 and WO2008/114021, etc.). Solid (e.g. dry powdered or lyophilized) compositions can be obtained by a process involving vacuum drying and freeze-drying (see e.g. WO2014/053571). It is envisaged that the pharmaceutical composition of the disclosure might comprise, in addition to the modified immune cells described herein, further biologically active agents, depending on the intended use of the pharmaceutical composition.

In some embodiments, the pharmaceutical composition is suitably buffered for human use. Suitable buffers include without limitation phosphate buffer (e.g. PBS), bicarbonate buffer and/or Tris buffer capable of maintaining a physiological or slightly basic pH (e.g. from approximately pH 7 to approximately pH 9). In some embodiments, the pharmaceutical composition can also be made to be isotonic with blood by the addition of a suitable tonicity modifier, such as glycerol.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the modified immune cells. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the modified immune cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the modified immune cells and the pharmaceutically acceptable excipient should be taken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

### IV. Kits and Articles of Manufacture

Also provided are kits, unit dosages, and articles of manufacture comprising any one of the modified immune cells, or the compositions (e.g. pharmaceutical composition) described herein. In some embodiments, a kit is provided which contains any one of the pharmaceutical compositions described herein and preferably provides instructions for its use. In some embodiments, the kit, in addition to the modified immune cell, further comprises a second cancer therapy, such as chemotherapy, hormone therapy, and/or immunotherapy. The kit(s) may be tailored to a particular cancer for an individual and comprise respective second cancer therapies for the individual.

The kits may contain one or more additional components, such as containers, reagents, culturing media, inducers, cytokines, buffers, antibodies, and the like to allow propagation or induction of the modified immune cell. The kits may also contain a device for local administration (such as intratumoral injection) of the pharmaceutical composition to a tumor site.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like. Some components of the kits may be packaged either in aqueous media or in lyophilized form.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g., from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### Examples

The examples below are intended to be purely exemplary of the invention and should therefore not be considered to limit the invention in any way. The following examples and detailed description are offered by way of illustration and not by way of limitation.

### Example 1: Design and Screening of Biologically Active Flagellin Fragments

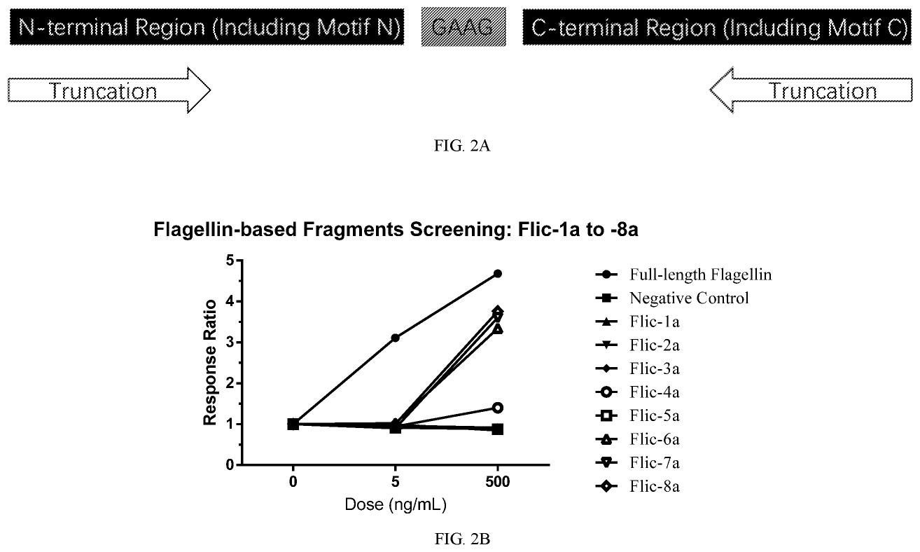

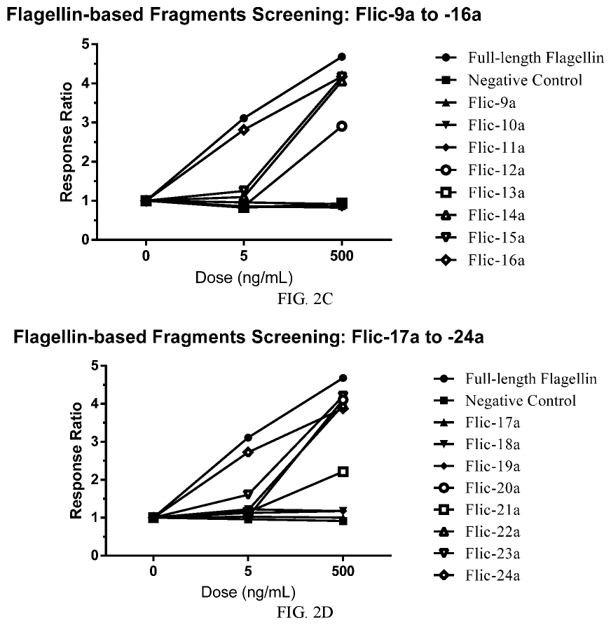

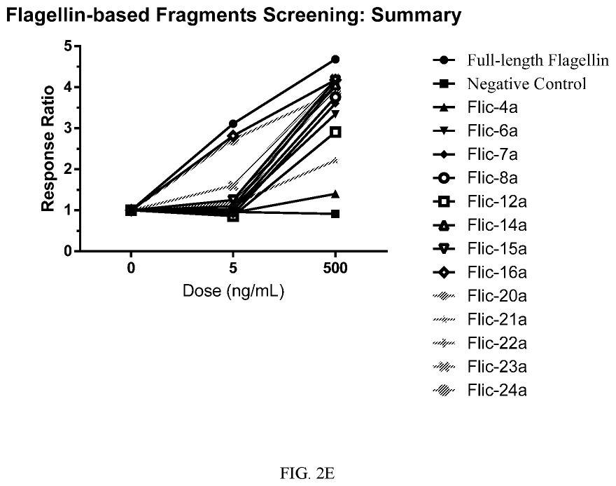

Twenty-four Salmonella typhimurium flagellin-based fragments (FIG. 2A, SEQ ID NOs: 9 to 32; see Table 2) were designed for screening using a secreted alkaline phosphatase (SEAP) reporter assay. These fragments were designed by progressively truncate the N-terminal and C-terminal regions of flagellin, while leaving Motifs C and N intact. In addition, the hypervariable region linking the C- and N-terminal regions of flagellin were replaced by a short GAAG linker (SEQ ID NO: 36) to reduce the size of the flagellin payload when used in combination with a chimeric antigen receptor. These peptides, together with full-length flagellin and negative control (Motif C/N-null flagellin), were synthesized by Genscript.

These flagellin-based fragments (also referred herein as FBFs) were screened in an NF-κB SEAP reporter assay. Briefly, the FBFs were incubated with stable engineered HEK293 cells expressing human TLR5 (puno1-ht1r5, Invivogen) and an NF-κB SEAP reporter (pnifty2-seap, Invivogen) in an HEK-blue detection media (hb-det3, Invivogen) at indicated concentrations overnight at 37° C. and 5% CO₂. Absorbance (i.e., OD) readings at 620 nm were taken and normalized against the basal reading to provide a response ratio for each sample. If an FBF activates TLR5, activation signal is expected to be transduced to the downstream NF-κB SEAP reporter, thereby resulting in a high response ratio. Using this assay, we identified two very potent FBFs, Flic 16a and Flic 24a, which displayed similar response ratios compared to full-length flagellin at very low concentrations (FIGS. 2B-2D). In addition, FBFs Flic 6a, 7a, 8a, 12a, 14a, 15a, 20a, 21a, 22a and 23a displayed at least 50% of TLR5 activation activity, compared to full-length flagellin, at higher concentrations (FIG. 2E).

In summary, we have identified a flagellin fragment (SEQ ID NO: 24) that is 207 amino acids in length, and displayed similar human TLR5 activation activity compared to full-length flagellin. Such fragments provide payloads with reduced size but intact signaling capability. As delivery vectors have size limits, the FBFs described herein enable combined delivery of a flagellin fragment with a chimeric antigen receptor via a single vector to immune cells as demonstrated in the following examples.

### Example 2: Design of Chimeric Antigen Receptors Armored with Biologically Active Flagellin Fragment-16a

A biologically active FBF, Flic-16a (SEQ ID NO: 24), was designed as a constitutively secreted FBF. The Flic16a-encoding sequence is combined with a conventional anti-BCMA CAR sequence via a self-cleaving P2A sequence in a plasmid to provide an armored CAR construct with Flic-16a. An armored CAR construct with full-length flagellin and an unarmored anti-BCMA CAR construct were also designed (FIG. 3A). A wide variety of antigen binding domain sequences are applicable for constructing the anti-BCMA CAR constructs disclosed herein. See, e.g., WO2017/025038, which is incorporated herein in its entirety. The sequences of the CAR constructs are shown below.

BCMA CAR amino acid sequence
SEQ ID NO: 33
MALPVTALLLPLALLLHAARPAVQLVESGGGLVQAGDSLRLTCTASGRAFS
TYFMAWFRQAPGKEREFVAGIAWSGGSTAYADSVKGRFTISRDNAKNTVYL
QMNSLKSEDTAVYYCASRGIEVEEFGAWGQGTQVTVSSGGGGSQVQLEESG
GGSVQAGGSLRLSCAYTYSTYSNYYMGWFREAPGKARTSVAIISSDTTITY
KDAVKGRFTISKDNAKNTLYLQMNSLKPEDSAMYRCAAWTSDWSVAYWGQG
TQVTVSSTSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQE
EDGCSCRFPEEEEGGCELRVKFSRSADAPAYQQGQNQLYNELNLGRREEYD
VLDKRRGRDPEMGGKPRRKNPQEGLYNELQKDKMAEAYSEIGMKGERRRGK
GHDGLYQGLSTATKDTYDALHMQALPPR
BCMA-CAR-Flic-16a FBF amino acid sequence
SEQ ID NO: 34
MALPVTALLLPLALLLHAARPAVQLVESGGGLVQAGDSLRLTCTASGRAFS
TYFMAWFRQAPGKEREFVAGIAWSGGSTAYADSVKGRFTISRDNAKNTVYL
QMNSLKSEDTAVYYCASRGIEVEEFGAWGQGTQVTVSSGGGGSQVQLEESG
GGSVQAGGSLRLSCAYTYSTYSNYYMGWFREAPGKARTSVAIISSDTTITY
KDAVKGRFTISKDNAKNTLYLQMNSLKPEDSAMYRCAAWTSDWSVAYWGQG
TQVTVSSTSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQE
EDGCSCRFPEEEEGGCELRVKFSRSADAPAYQQGQNQLYNELNLGRREEYD
VLDKRRGRDPEMGGKPRRKNPQEGLYNELQKDKMAEAYSEIGMKGERRRGK
GEIDGLYQGLSTATKDTYDALHMQALPPRGSGATNFSLLKQAGDVEENPGP
MDWTWILFLVAAATRVHSRINSAGISIAQTTEGALNEINNNLQRVRELAVQ
SANSTNSQSDLDSIQAEITQRLNEIDRVSGQTQFNGVKVLAQDNTLTIQVG
ANDGETIDIDLKQINSQTLGLDTLNGAAGATTTENPLQKIDAALAQVDTLR
SDLGAVQNRFNSAITNLGNTVNNLTSARSRIEDSDYATEVSNMSRAQILQQ
AGTSVLAQANQVPQNVLSLLR
BCMA-CAR-Full-length Flagellin amino acid sequence
SEQ ID NO: 35
MALPVTALLLPLALLLHAARPAVQLVESGGGLVQAGDSLRLTCTASGRAFS
TYFMAWFRQAPGKEREFVAGIAWSGGSTAYADSVKGRFTISRDNAKNTVYL
QMNSLKSEDTAVYYCASRGIEVEEFGAWGQGTQVTVSSGGGGSQVQLEESG
GGSVQAGGSLRLSCAYTYSTYSNYYMGWFREAPGKARTSVAIISSDTTITY
KDAVKGRFTISKDNAKNTLYLQMNSLKPEDSAMYRCAAWTSDWSVAYVVGQ
GTQVTVSSTSTTTPAPRPPTPAPTIASQPLSLRPEACRPAAGGAVHTRGLD
FACDIYIWAPLAGTCGVLLLSLVITLYCKRGRKKLLYIFKQPFMRPVQTTQ
EEDGCSCRFPEEEEGGCELRVKFSRSADAPAYQQGQNQLYNELNLGRREEY
DVLDKRRGRDPEMGGKPRRKNPQEGLYNELQKDKMAEAYSEIGMKGERRRG
KGEIDGLYQGLSTATKDTYDALHMQALPPRGSGATNFSLLKQAGDVEENPG
PMDWTWILFLVAAATRVHSMAQVINTNSLSLLTQNNLNKSQSALGTAIERL
SSGLRINSAKDDAAGQAIANRFTANIKGLTQASRNANDGISIAQTTEGALN
EINNNLQRVRELAVQSANSTNSQSDLDSIQAEITQRLNEIDRVSGQTQFNG
VKVLAQDNTLTIQVGANDGETIDIDLKQINSQTLGLDTLNVQQKYKVSDTA
ATVTGYADTTIALDNSTFKASATGLGGTDQKIDGDLKFDDTTGKYYAKVTV
TGGTGKDGYYEVSVDKTNGEVTLAGGATSPLTGGLPATATEDVKNVQVANA
DLTEAKAALTAAGVTGTASVVKMSYTDNNGKTIDGGLAVKVGDDYYSATQN
KDGSISINTTKYTADDGTSKTALNKLGGADGKIEVVSIGGKTYAASKAEGH
NFKAQPDLAEAAATT1ENPLQKIDAALAQVDTLRSDLGAVQNRFNSAITNL
GNTVNNLTSARSRIEDSDYATEVSNMSRAQILQQAGTSVLAQANQVPQNVL
SLLR

### Example 3: Viral Transfection and Viral Particle Production

Lentiviral vector, pLVX-Puro (Clontech#632164), was modified by replacing its built-in promoter with human elongation factor 1α promoter (hEF1α) to provide PLVX-EF1A. Each of the CAR constructs (i.e., armored CAR with Flic 16a, armored CAR with full-length flagellin, and an unarmored CAR construct) of Example 2 was cloned into the lentiviral vector by EcoRI and BamHI digestion, which removed the puromycin resistance gene. The resulting lentiviral vectors encoding the CAR constructs were packaged into viral particles as described below.

Briefly, a lentivirus packaging plasmid mixture including pMDLg/pRRE (Addgene#12251), pRSV-Rev (Addgene#12253), and pMD2.G (Addgene#12259) was pre-mixed with a PLVX-EF1A vector encoding a CAR construct at a pre-optimized ratio with polyethylenimine (PEI), mixed properly, and incubated at room temperature for 5 minutes. The transfection mix was added dropwise to 293-T cells with gentle mixing. Transfected 293-T cells were incubated overnight at 37° C. and 5% CO₂. Twenty-four hours post transfection, supernatants were collected and centrifuged at 4° C., 500 g for 10 minutes to remove any cellular debris. Centrifuged supernatants were filtered through a 0.45 μm PES filter to concentrate the viral supernatants by ultracentrifugation. After centrifugation, the supernatants were carefully removed and the virus pellets were rinsed with pre-chilled DPBS buffer. The concentration of virus was measured. Virus was aliquoted and stored at −80° C. Viral titer was determined by functional transduction of a T cell line.

### Example 4: Immune Cell Preparation

Leukocytes were collected in R10 medium and mixed with 0.9% NaCl solution at a 1:1 (v/v) ratio. 3 mL of Lymphoprep medium was added to a 15 mL centrifuge tube containing 3 mL of leukocyte slowly to provide 6 mL of diluted lymphocyte mix. The lymphocyte mix was centrifuged at 800 g for 30 minutes without brake at 20° C. Lymphocyte buffy coat was then collected with a 200 μL pipette. The harvested fraction was diluted at least 6 fold using 0.9% NaCl or R10 medium to reduce the density of the solution. The harvested fraction was then centrifuged at 250 g for 10 minutes at 20° C. The supernatant was aspirated completely, and 10 mL of R10 medium was added to the cell pellet. The mixture was further centrifuged at 250 g for 10 minutes at 20° C. The supernatant was again aspirated. 2 mL of R10 medium was pre-warmed at 37° C., which was then added to the cell pellet together with 100 IU/mL IL-2. The cell pellet was subsequently gently re-suspended to provide a PBMC sample. Number of cells in the PBMC sample was counted. Human T cells were purified from the PBMC sample using the Miltenyi Pan T cell isolation kit (Cat#130-096-535) to provide alpha/beta T cells.

The alpha/beta T cells were subsequently pre-activated for 48 hours using a human T cell Activation/Expansion kit (Milteny#130-091-441). One loaded anti-Biotin MACSiBead Particle was used for every two cells (i.e., bead-to-cell ratio of 1:2).

Gamma/delta T cells were prepared by addition of 5 μM Zoledronate and 1000 IU/mL IL-2 to PBMCs and cultured for 14 days with periodical change of media supplemented with 1000 IU/mL IL-2. Alternatively, gamma/delta T cells were isolated from PBMCs or umbilical cord blood (UCB) and then stimulated by anti-gamma/delta TCR antibody and anti-CD3 antibody (OKT3) followed by co-incubation of K562-based artificial antigen-presenting cells (aAPCs) at a 1:2 ratio for at least 10 days.

### Example 5: T Cell Transduction

### Transduction of Alpha/Beta T Cells

The pre-activated alpha/beta T cells were collected and re-suspended in 1640 medium containing 300 IU/mL IL-2. Lentiviral particles comprising vectors encoding each of the CAR constructs of Example 2 were diluted to a multiplicity of infection (MOI) of 5 with the same medium, and used to infect 10⁶ activated alpha/beta T cells. The pre-activated T cells were transduced with stock lentiviruses in the presence of 8 μg/ml polybrene with centrifugation at 1000 g, 32° C. for 1 hours. The transduced cells were then transferred to a cell culture incubator to allow transgene expression under suitable conditions. The following day, the transduced cells were centrifuged and resuspended with fresh media. Cells density was measured every other day, and fresh media were added to allow continued T cell expansion.

### Transduction of Gamma/Delta T Cells

PBMCs were isolated by density centrifugation (lymphoprep) from leukapheresis material and cryopreserved. PBMCs were recovered and activated with zoledronic acid (5 μM) in cell culture media AIM-V supplemented with IL-2 (1000 IU/ml) and 5% human AB serum and kept in a humidified chamber (37° C., 5% CO₂). Forty-eight hours post-PBMC activation, cells were transduced with lentiviral particles comprising vectors encoding each of the CAR constructs at an MOI of 5 with 5 pg/ml polybrene. Such transduction procedure was repeated the next day followed by replenishment of fresh media containing IL-2 (1000 IU/ml) the day after the second transduction. Cells were cultured in AIM-V supplemented with IL-2 (1000 IU/ml) in a humidified chamber with periodical change of media as determined by the pH of the culture media to allow further T cell expansion. Cells were harvested 10 days post-transduction and the total number, purity and transduction efficiency of the cells were determined. Cells were further enriched using a negative TCRγ/δ+ T cell isolation kit (Miltenyi Biotec) before future applications or cryopreservation.

### Example 6: Quantification of Chimeric Antigen Receptor Expression

On day 3 and onwards (typically day 3, 7 and 14) post-transduction, cells were evaluated for expression of the CARs of Example 2 by flow cytometry. An aliquot of cells was collected from the culture, washed, pelleted, and resuspended in 50-100 μl of diluted antibody (eBioscience Anti-Mouse TCR beta PE and anti-CAR antibody) at 1:100 dilution in PBS+0.5% FBS. Cell were then incubated at 4° C. for 30 minutes. Viability dye, eFluor780 or SYTOX Blue, was also added to the cells according to the manufacturer's instructions. Post-incubation, cells were washed twice in PBS and resuspended in 100 to 2000 PBS for analysis. The mean fluorescence of each sample was quantified by flow cytometry.

For anti-BCMA sdAb staining, cells were stained with Alexa Fluor 488-labeled mouse-anti-camel sdAb antibodies (Genscript). Results of all flow cytometry experiments were analyzed using FlowJo (Tree Star, Inc.).

### Example 7: Cytotoxicity Assay

### Cytotoxicity of Alpha/Beta T Cells Expressing Full-Length Flagellin or FBF-Armored CAR

Cytotoxicity of αβT cells expressing armored CAR constructs with full-length flagellin or Flic-16a FBF (FIG. 3A), as well as control αβT cells expressing an unarmored CAR construct, was determined in a 20 hour co-culture assay. Briefly, the effector cells (i.e., αβT cells) were collected by centrifugation, then diluted to the desired concentrations with 1640 phenol red free medium (Invitrogen) supplemented with 2% heat inactivated FBS (Invitrogen). The target cells, H929 (a human plasmacytoma/myeloma cell line), exhibited decent expression of target antigen BCMA. The effector cells were co-cultured with the target cells at different effector to target ratios (E:T=2:1, 1:1. 0.5:1 and 0.25:1) at 37° C. for 20 hours in a 96-well plate. Wells containing assay buffer only (1640 phenol red free medium plus 2% hiFBS), target cell only (T), effector cell only (E) and maximum lysis of target cell (1% solution of tritonX-100) were included as control conditions. Each condition was performed in triplicate, and the cytotoxicity of effector cells was detected using a Lactate Dehydrogenase (LDH) assay kit (Roche). After completion of the 20-hour co-culture, the assay plate was centrifuged, and supernatant was collected in a new 96-well plate. The supernatant plate was diluted with an equal volume of the LDH assay reagent according to the manufacture's manual. The assay plate was incubated for about 30 minutes at 15° C.-25° C. The absorbance of the plate was measured at 492 nm and 650 nm using Flexstation reader (Molecular Devices) and cytotoxicity was calculated as previously described.

As shown in FIG. 3B, at every E:T ratio, both full-length flagellin and Flic-16a FBF-armored CAR expressing αβ T cells displayed higher efficacy against target H929 cells than unarmored CAR-αβT cells. Furthermore, Flic-16a-FBF-armored CAR-αβT cells displayed similar efficacy compared to full-length flagellin-armored CAR-αβT cells at higher E:T ratios. Cytotoxicity of gamma/delta T cells expressing full-length flagellin or FBF-armored CAR

γδT cells were transduced with lentiviral particles comprising vectors that encode full-length flagellin or Flic-16a FBF-armored CAR construct, or control unarmored CAR. Cytotoxicity of the effector γδT cells was assessed seven days post-transduction. Briefly, transduced or non-transduced γδT cells were incubated with BCMA positive target cell line, H929, and the cytotoxic effects of γδT cells were evaluated using an LDH assay kit (Roche) as described above.

As shown in FIG. 3C, unlike CAR-αβT cells, FBF Flic-16a-armored CAR-γδT cells showed similar efficacy compared to full-length flagellin-armored CAR-γδT cells at all E:T ratios tested. In both cases, a flagellin armor provided clear benefits because increased cytotoxic effects were observed in full-length flagellin- or Flic-16a FBF-armored CAR-αβ T cells or CAR-γδT cells compared to the unarmored CAR-T counterparts.

### Example 8: Cytokine Release

43 or γδT cells transduced with lentiviral particles comprising vectors that encode full-length flagellin or Flic-16 FBF-armored CAR construct, or control unarmored CAR construct were co-cultured with BCMA-positive H929 cells at a ratio of 1:1 for 48 hours at 37° C. Supernatants of the co-cultures were collected to analyze cytokine release by the effector cells using the following kits: Human IFN gamma kit (Cisbio, Cat#62HIFNGPEH), Human TNF alpha kit (Cisbio, Cat#62HTNFAPEH); and Human IL2 kit (Cisbio, Cat#62HIL02PEH). Briefly, the cell supernatants and a standard were dispensed directly into an assay plate for cytokine detection using HTRF® reagents. Antibodies labeled with the HTRF donor and acceptor were pre-mixed and added to the samples in a single dispensing step.

An ELISA standard curve was generated according to the 4 Parameter Logistic (4PL) curve. The standard curve regression method enables accurate measurement of the concentration of an unknown sample across a wider range of concentrations than linear regression. Thus, the 4PL regression method is suitable for analysis of biological systems such as cytokine release.

IFN-γ, TNF-α and IL-2 Release by Alpha/Beta T Cells Expressing Full-Length Flagellin- or FBF-Armored CAR Co-Cultured with Target Cells

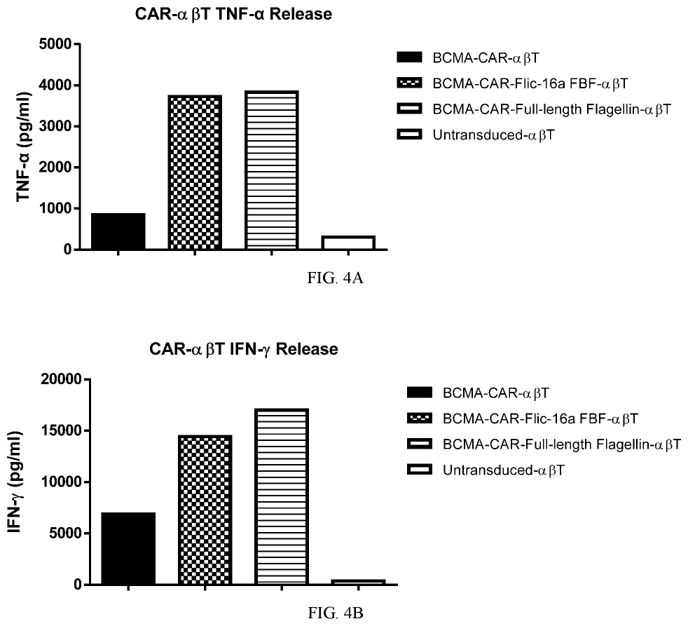

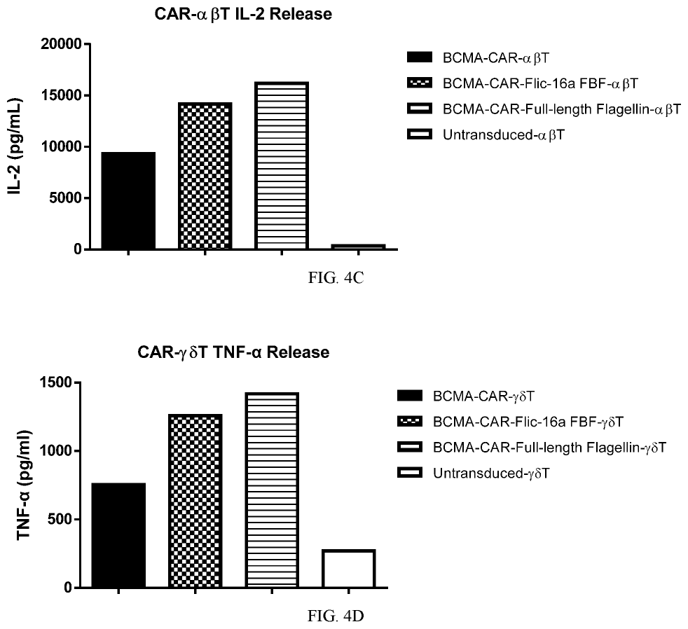

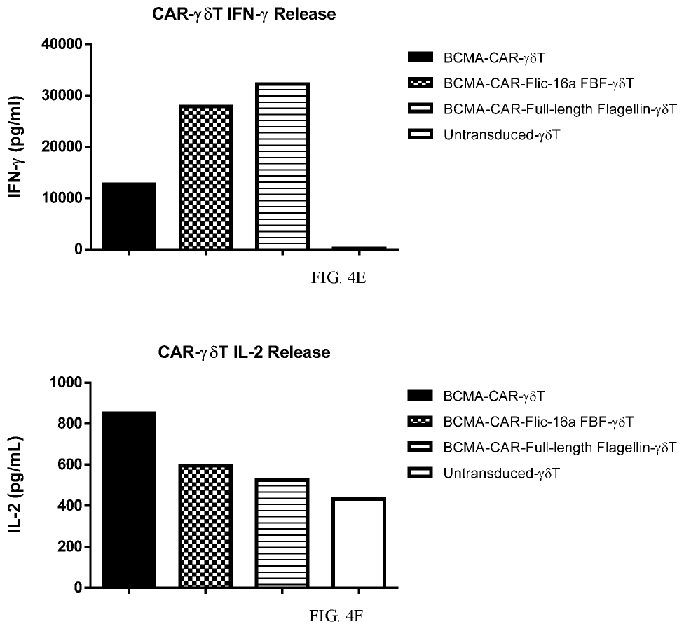

As shown in FIGS. 4A-4C, both full-length flagellin-armored and Flic-16a FBF-armored CAR-αβT cells showed significantly higher IFN-γ, TNF-α and IL-2 release levels than unarmored CAR-αβT cells when co-cultured with target cells. In addition, full-length flagellin-armored CAR-T cells secreted slightly higher levels of cytokines than the Flic-16a FBF-armored CAR-αβT cells. Taken together, flagellin-armored CAR-αβT cells display a potent cytokine release profile, which correlates with the high anti-tumor cytotoxicity observed and described in Example 7.

IFN-γ, TNF-α and IL-2 Release by Gamma/Delta T Cells Expressing Full-Length Flagellin- or FBF-Armored CAR Co-Cultured with Target Cells

The cytokine release profiles of flagellin-armored CAR-γδT cells are similar to those of flagellin-armored CAR-αβT cells. As shown in FIGS. 4D-4F, both full-length flagellin- and Flic-16a FBF-armored CAR-γδT cells showed significantly higher IFN-γ and TNF-α release levels, albeit a slightly lower level of IL-2 release, compared to unarmored CAR-γδT cells when co-cultured with target cells. Also, full-length flagellin-armored CAR-γδT cells secreted slightly higher levels of cytokines, except for IL-2, than the Flic-16a FBF-armored CAR-γδT cells. Taken together, flagellin-armored CAR-γδT cells display a potent cytokine release profile, which correlates with the high anti-tumor cytotoxicity observed and described in Example 7.

1. A modified immune cell comprising a first heterologous nucleic acid sequence encoding a flagellin polypeptide comprising a flagellin protein or a fragment thereof, wherein the flagellin polypeptide upon expression is capable of binding to a toll-like receptor.

2-3. (canceled)

4. The modified immune cell of claim 1, wherein the flagellin polypeptide comprises an N-terminal domain comprising Motif N of a flagellin protein and a C-terminal domain comprising Motif C of the flagellin protein, wherein the N-terminal domain and the C-terminal domain are fused to each other via a peptide linker.

5. The modified immune cell of claim 1, wherein the flagellin polypeptide comprises all or a portion of a flagellin protein from S. typhimurium, S. muenchen, or E. coli.

6. (canceled)

7. The modified immune cell of claim 1, wherein the flagellin polypeptide comprises an amino acid sequence having at least about 85% sequence identity to an amino acid sequence selected from the group consisting of SEQ ID NOs: 12, 14-16, 20, 22-24, and 28-32.

8. (canceled)

9. The modified immune cell of claim 1, wherein the toll-like receptor is selected from the group consisting of TLR4, TLR5, TLR11, TLR2, TLR3, and TLR9.

10. The modified immune cell of claim 1, wherein the flagellin polypeptide is membrane-bound.

11. The modified immune cell of claim 10, wherein the flagellin polypeptide is bound to the membrane via a glycosylphosphatidylinositol (GPI) linker or a transmembrane domain.

12. (canceled)

13. The modified immune cell of claim 11, wherein the flagellin polypeptide is bound to the membrane via a transmembrane domain, and wherein the transmembrane domain is derived from a molecule selected from the group consisting of CD8, CD4, CD28, 4-1BB, CD80, CD86, CD152 and PD1.

14. The modified immune cell of claim 11, wherein the flagellin polypeptide is bound to the membrane via a transmembrane domain, and wherein the flagellin polypeptide further comprises an intracellular signaling domain.

15. The modified immune cell of claim 14, wherein the intracellular signaling domain is derived from a co-stimulatory molecule selected from the group consisting of CD27, CD28, 4-1BB, OX40, CD30, CD40, CD3, LFA-1, CD2, CD7, LIGHT, NKG2C, B7-H3, Ligands of CD83 and combinations thereof.

16. The modified immune cell of claim 1, wherein the flagellin polypeptide is secreted by the modified immune cell.

17. The modified immune cell of any claim 1, wherein the modified immune cell is selected from the group consisting of a tumor-infiltrating T cell, a dendritic cell (DC)-activated T cell, a cytotoxic T cell, a helper T cell, a natural killer (NK) cell, an NK-cell, an iNK-T cell, an NK-T like cell, an αβT cell and a δδT cell.

18. The modified immune cell of claim 17, wherein the modified immune cell is a cytotoxic T cell.

19. (canceled)

20. The modified immune cell of claim 1, wherein the modified immune cell comprises a second heterologous nucleic acid sequence encoding an engineered receptor.

21. The modified immune cell of claim 20, wherein the engineered receptor is a chimeric antigen receptor (CAR), a modified T-cell receptor (TCR), or a T-cell antigen coupler (TAC) receptor.

22. The modified immune cell of claim 20, wherein the engineered receptor is a CAR, and wherein the CAR is an anti-BCMA CAR.

23-26. (canceled)

27. A method of producing a modified immune cell, comprising: introducing into a precursor immune cell a first nucleic acid sequence encoding a flagellin polypeptide comprising a flagellin protein or a fragment thereof, wherein the flagellin polypeptide upon expression is capable of binding to a toll-like receptor.

28-37. (canceled)

38. A pharmaceutical composition comprising the modified immune cell of claim 1, and a pharmaceutically acceptable carrier.

39. A method of treating a disease in an individual, comprising administering to the individual an effective amount of the pharmaceutical composition of claim 38.

40-41. (canceled)

42. An engineered flagellin polypeptide comprising an amino acid sequence having at least about 85% sequence identity to an amino acid sequence selected from the group consisting of SEQ ID NOs: 12, 14-16, 20, 22-24, and 28-32.
